SEQUENCE LISTING (14 sequences)

SEQ ID NO: 1 - DNA, 20 bp, Mus musculus
catcgaagctcaagatatgg

SEQ ID NO: 2 - DNA, 20 bp, Mus musculus
gatttccattgatgatggtg

SEQ ID NO: 3 - DNA, 20 bp, Mus musculus
acacagagactggcaacatc

SEQ ID NO: 4 - DNA, 20 bp, Mus musculus
gagcaatccctgaatagtcc

SEQ ID NO: 5 - DNA, 20 bp, Mus musculus
tggacacatctcctaggttg

SEQ ID NO: 6 - DNA, 20 bp, Mus musculus
tagagaagagtcgggagacc

SEQ ID NO: 7 - DNA, 20 bp, Mus musculus
ccagtatgactccactcacg

SEQ ID NO: 8 - DNA, 20 bp, Mus musculus
gactccacgacatactcagc

SEQ ID NO: 9 - DNA, 29 bp, Mus musculus
ctctgaacaggtagtcgatagcgacagac

SEQ ID NO: 10 - DNA, 26 bp, Mus musculus
cggagacactgcctgtgttctcattg

SEQ ID NO: 11 - DNA, 24 bp, Mus musculus
gcatcgccttctatcgccttcttg

SEQ ID NO: 12 - DNA, 20 bp, Mus musculus
ggaacttgtgcaggttggat

SEQ ID NO: 13 - DNA, 20 bp, Mus musculus
cagtgcaagctccaagatga

SEQ ID NO: 14 - DNA, 20 bp, Mus musculus
atactttctcggcaggagca

(19) United States
(12) Patent Application Publication
(10) Pub. No.: US 2010/0269180 A1
(43) Pub. Date: Oct. 21, 2010

(54) METHODS, COMPOSITIONS AND TRANSGENIC MODELS RELATED TO THE INTERACTION OF T-CADHERIN AND ADIPONECTIN

(76) Inventors: Barbara Ranscht, Del Mar, CA (US); Martin Denzel, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/762,163

(22) Filed: Apr. 16, 2010

Related U.S. Application Data
(60) Provisional application No. 61/170,493, filed on Apr. 17, 2009.
(60) Provisional application No. 61/299,858, filed on Jan. 29, 2010.
(60) Provisional application No. 61/300,246, filed on Feb. 1, 2010.

(51) Int. Cl. G01N 33/00; A61K 38/02; A61K 38/22; A61P 9/00; A61P 9/10; C12Q 1/02; A01K 67/027

(52) U.S. Cl. 800/3; 514/16.4; 514/9.7; 435/29; 800/18

(57) ABSTRACT

Disclosed are methods, compositions and transgenic models related to the interaction of T-cadherin and adiponectin.

### RELATED APPLICATIONS

This application claims priority under 35 U.S.C. §119(e) to U.S. Provisional Patent Application Ser. No. 61/170,493, filed on Apr. 17, 2009, U.S. Provisional Patent Application Ser. No. 61/299,858, filed on Jan. 29, 2010, and U.S. Provisional Patent Application Ser. No. 61/300,246, filed on Feb. 2, 2010, each of which is incorporated herein by reference in its entirety.

### STATEMENT REGARDING FEDERALLY SPONSORED R&D

The T-cadherin knockout mice described herein were made in part with United States government support under NIH grant number HD 25938. Part of the invention described herein formed the basis for federal funding through NIH grant R21 HL102680, which was awarded on Apr. 1, 2010. The U.S. government has certain rights in this invention.

### BACKGROUND OF THE INVENTION

1. Field of the Invention

The present invention is in the field of methods, compositions and transgenic models related to the interaction of T-cadherin and adiponectin. In some embodiments, methods of conferring a cardioprotective effect, modulating cardiomyocyte growth, treating or ameliorating a cardiovascular condition, screening for a candidate compound, or treating cardiac hypertrophy or aortic rupture are provided.

2. Description of the Related Art

Cardiac hypertrophy is the enlargement of cardiomyocyte cell volume in postnatal development. Cardiac hypertrophy is induced by physiological stimuli such as physical activity or through pathological stressors such as hypertension, aortic stenosis, ischemic heart disease, valvular insufficiency, infectious agents or mutations in sarcomeric genes (1). According to LaPlace's Law, the load on the myocardium is given as (pressure*radius)/(2*wall thickness). Thus, an increase in ventricular pressure during hemodynamic overload can be compensated for by an increase in wall thickness. As cardiomyocytes are terminally differentiated cells that cannot divide, growth in cell volume is the key adaptive mechanism to react to pressure overload (1). This remodeling is achieved by parallel addition of sarcomeres in existing muscle fibers, resulting in increased myocyte width. This process is termed “concentric hypertrophy,” which is defined as increased ratio of wall thickness/chamber dimension. Pathologic hypertrophy can temporarily preserve the heart's pump function, but a prolonged hypertrophic state leads to dilated cardiomyopathy—and ultimately to heart failure.

Researchers have investigated what distinguishes “good” adaptive hypertrophy (e.g., that observed in athletes) from “bad hypertrophy” that leads to decompensation. Various intracellular signaling cascades have been studied, and the identification of protective versus deleterious pathways is the subject of intense research. For example, Notchl limits hypertrophy of cardiomyocytes and thus protects against pathological remodeling of the myocardium (2). Activated c-Jun N-terminal kinase (MK) and p38 signaling, on the other hand, are strongly implicated in the development of pathological hypertrophy (3).

The serine/threonine protein kinase Akt is also involved in cardiac hypertrophy and regulates growth, survival and metabolism (4). Activation of the Akt/mammalian target of rapamycin (Akt/mTOR) pathway was found in physiologically hypertrophied hearts of mice trained on a treadmill, while surgically induced aortic constriction led to pathological hypertrophy that was accompanied by Akt inactivation (5).

The role of increased Ca²⁺ signaling in conditions of pressure overload may also contribute to cardiac hypertrophy. Given the constant rhythmic fluctuations of intracellular Ca²⁺ in myocytes, this is a particularly challenging research question. One study implicates an important role of CaM kinase II in the development of pathologic hypertrophy during pressure overload (6).

Angiogenesis is another key factor in maintaining myocardial function. Increased cardiac workload triggers hypertrophy, which leads to hypoxia and to the activation of Hif-1, which subsequently triggers the expression of pro-angiogenic factors and angiogenesis. Hypertrophic growth depends on angiogenesis. This coordinated growth of cardiomyocytes and vasculature maintains cardiac function during pressure overload for about 14 days after experimental increase of cardiac afterload. Prolonged pressure overload, however, leads to accumulation of p53, which shuts down Hif-1, and thus inhibits angiogenesis. Inhibition of p53 maintains responsive vascular growth and cardiac function. Thus, the p53-dependent reduction in angiogenesis appears to be a key factor in the transition from cardiac hypertrophy to heart failure (6).

AMP-activated protein kinase (AMPK) is the key regulator of metabolism in the heart (7). Activated in conditions of energy depletion, AMPK halts anabolic processes such as glycogen, fatty acid and protein synthesis, while increasing ATP-producing processes, such as glycolysis and fatty acid oxidation. During myocardial stress, AMPK becomes activated and has been shown to protect the heart from pathological hypertrophy. Importantly, the cardioprotective effects of adiponectin are mediated in part through AMPK (8).

Adiponectin is a global regulator of metabolism and plays important roles in the cross talk between the adipose tissue and numerous organs, such as liver, brain, muscle, vasculature and the heart. Understanding the regulation and function of adiponectin is important, as there is good evidence that adiponectin loss in obesity is linked to metabolic syndrome, whose primary clinical outcome is cardiovascular disease. Obesity is strongly associated with cardiovascular disorders, including myocardial ischemia, atherosclerosis and inflammation. Adiponectin deficiency is also an independent risk factor for Type 2 diabetes, hypertension and coronary artery disease. Paradoxically, concentrations of the fat-derived plasma protein adiponectin are significantly reduced in obese individuals. In these individuals, adiponectin is downregulated, and is therefore implicated in the etiology of metabolic syndrome (9). However, the precise mechanism by which adiponectin influences the physiology of its target tissues is poorly understood.

Adiponectin has numerous beneficial metabolic and cardiovascular properties (10). Adiponectin is anti-inflammatory, anti-atherosclerotic and protects from neointima formation after endothelial injury. Adiponectin's anti-inflammatory role has been described in many reports. For example, TNFα concentrations are increased during hypoadiponectinemia, and adiponectin has been shown to protect from inflammation in an experimental stroke model in rats (12). Adiponectin further maintains vascular homeostasis by activation of endothelial nitric oxide synthase, which results in the release of nitric oxide (13). Nitric oxide release, in turn, inhibits leukocyte adhesion to the vascular endothelium and scavenges superoxide anions, thus inhibiting inflammatory processes.

In the heart, adiponectin exerts protective effects, such as decreased infarct size or improved outcome of ischemia/reperfusion studies in mice. Moreover, adiponectin blunts cardiac hypertrophy after pressure overload. Once again, evidence suggests that these cardioprotective effects of adiponectin are mediated through AMPK (8).

T-cadherin is a glycophosphatidylinositol (GPI)-linked glycoprotein and putative adiponectin receptor. An unusual member of the cadherin family of proteins, T-cadherin is involved in the functions of adiponectin. This adipocyte-produced cytokine possesses beneficial functions in normal physiology and is suggested to exert protective roles in metabolic and vascular diseases.

Although T-cadherin is abundantly expressed in the heart, its cardiac function is largely unknown (14). Studies have revealed that T-cadherin can be found in Triton-insoluble membrane domains of the sarcolemma, and suggest that T-cadherin is located in plasma membrane “rafts” of cardiac myocytes (14, 15).

### SUMMARY OF THE INVENTION

The present invention pertains to methods, compositions and transgenic models related to the interaction of T-cadherin and adiponectin.

One embodiment of the invention is a method of conferring a cardioprotective effect in a mammal, comprising administering a compound to the mammal, where the compound is known to interact with a pro-peptide form of T-cadherin.

Another embodiment of the invention is a method of modulating cardiomyocyte growth, comprising administering to a subject a compound known to interact with the pro-peptide portion of T-cadherin.

Another embodiment of the invention is a method of treating or ameliorating a cardiovascular condition, comprising administering a compound known to bind to a pro-peptide form of T-cadherin.

Another embodiment of the invention is a method of screening a candidate compound for the ability to confer a cardioprotective effect, comprising: providing a candidate compound to a cell; determining whether the candidate compound is capable of stimulating the activity of a T-cadherin protein; and for a test compound capable of stimulating the activity of T-cadherin, determining whether the test compound is capable of, or likely to be capable of, conferring a cardioprotective effect in the cell.

Another embodiment of the invention is a method of treating cardiac hypertrophy in a mammal, the method comprising identifying a mammal suffering from cardiac hypertrophy and providing to the mammal a therapeutically effective amount of an adiponectin polypeptide or fragment thereof.

Another embodiment of the invention is a method of treating aortic rupture in a mammal, the method comprising identifying a mammal suffering from aortic rupture and providing to the mammal a therapeutically effective amount of an adiponectin polypeptide or fragment thereof.

Another embodiment of the invention is a method for the treatment and/or prophylaxis of a condition characterized by cardiac hypertrophy in a mammal, the method comprising modulating the interaction of adiponectin and T-cadherin in a mammal, where upregulating the activity of adiponectin to a functionally effective level upregulates the interaction of adiponectin and T-cadherin in the mammal.

Another embodiment of the invention is a method for the treatment and/or prophylaxis of a condition characterized by aortic rupture in a mammal, the method comprising modulating the interaction of adiponectin and T-cadherin in a mammal, where upregulating the activity of adiponectin to a functionally effective level upregulates the interaction of adiponectin and T-cadherin.

Another embodiment of the invention is a method of identifying a molecule that transduces an adiponectin signal to the interior of a cell comprising: providing a candidate molecule to a cell; and determining whether the candidate molecule is capable of stimulating the activity of T-cadherin, where if the candidate molecule is capable of stimulating the activity of T-cadherin it is identified as transducing an adiponectin signal.

Another embodiment of the invention is a genetically modified mouse comprising a disrupted T-cadherin gene, where the genetically modified mouse is homozygous for the disrupted T-cadherin gene, and where the genetically modified mouse exhibits an increase in cardiac hypertrophy compared to a mouse that does not have the disrupted T-cadherin gene.

Another embodiment of the invention is a genetically modified mouse comprising a disrupted adiponectin gene and a disrupted T-cadherin gene, where the genetically modified mouse is homozygous for the disrupted adiponectin gene and the disrupted T-cadherin gene.

Another embodiment of the invention is a method of using a genetically modified mouse to identify a compound that confers a cardioprotective effect comprising: providing one the genetically modified mice described above; contacting the genetically modified mouse with a compound; determining whether the compound is capable of stimulating the activity of a T-cadherin protein; and for a candidate compound capable of stimulating the activity of T-cadherin, determining whether the candidate compound is capable of, or likely to be capable of, conferring a cardioprotective effect in the mouse.

Another embodiment of the invention is a composition capable of stimulating the activity of a T-cadherin protein, where the composition comprises an adiponectin polypeptide or fragment thereof.

Another embodiment of the invention is a composition for the prevention, amelioration or reversal of a cardiovascular condition, where the composition is configured to upregulate a posttranslational modification of T-cadherin.

In some embodiments of the invention, the invention comprises one or more of the following methods: a method where the interaction comprises the binding of the compound to the pro-peptide form of T-cadherin; a method where the interaction comprises the sequestration of the pro-peptide form of T-cadherin to the surface of a cell in a mammal; a method where the cardioprotective effect comprises the prevention, amelioration or reversal of a condition selected from the group consisting of: cardiac hypertrophy, aortic rupture, and myocardial infarct; a method where the cardiovascular condition is selected from the group consisting of: cardiac hypertrophy, aortic rupture, and myocardial infarct; a method where determining whether the candidate compound is capable of stimulating the activity of a T-cadherin protein comprises measuring the level of AMP-activated protein kinase (AMPK) phosphorylation in the cell; a method where determining a stimulated activity of the T-cadherin protein comprises detecting a posttranslational modification of the T-cadherin protein; a method where the candidate compound interacts with a pro-peptide form of the T-cadherin protein; a method where the candidate compound interacts with a mature form of the T-cadherin protein; a method where the candidate compound is a compound from a peptide library; a method where the cardioprotective effect comprises the prevention, amelioration or reversal of cardiac hypertrophy in a mammal; a method where the cardiac hypertrophy has been detected or predicted using a measurement in heart size, heart weight to body weight ratio, diastolic left-ventricular posterior wall thickness, or myofiber area; a method where the aortic rupture has been detected or predicted using a measurement in macrophage infiltration into the aortic wall; a method where the aortic rupture has been detected or predicted using a measurement in a pro-inflammatory marker; and a method where the compound is an adiponectin polypeptide or fragment thereof; a method where the condition is selected from the group consisting of: hypertension, aortic stenosis, ischemic heart disease, valvular insufficiency, infectious agents or mutations in sarcomeric genes; a method further comprising subjecting the mouse to transaortic constriction (TAC) to determine whether the candidate compound is capable of, or likely to be capable of, conferring a cardioprotective effect in the mouse; and a method where determining a stimulated activity of the T-cadherin protein comprises detecting a posttranslational modification of the T-cadherin protein.

In some embodiments of the invention, the invention comprises the following: a mouse where the disrupted T-cadherin gene comprises a tissue-specific null mutation; a mouse where the disrupted adiponectin gene comprises a tissue-specific null mutation; a mouse where the increase in cardiac hypertrophy is detected using a measurement in heart size, heart weight to body weight ratio, diastolic left-ventricular posterior wall thickness, myofiber area, macrophage infiltration into the aortic wall, or a pro-inflammatory marker; and a mouse where the genetically modified mouse exhibits an increase in cardiac hypertrophy compared to a mouse that does not have the disrupted adiponectin gene or the disrupted T-cadherin gene.

In some embodiments of the invention, the invention comprises the following: a composition that comprises a region capable of binding with a pro-peptide form of the T-cadherin protein; and a composition that comprises a region capable of binding with a mature form of the T-cadherin protein.

### BRIEF DESCRIPTION OF THE DRAWINGS

FIG. 1. Transaortic Constriction (TAC)

(A) The heart during TAC. A banding site is observed on the transaortic arch. (B) Schematic representation of a heart cross section during the phases of hypertrophic response to TAC. Adapted from Chien et al., 1989 (16).

FIG. 2. T-cadherin and Adiponectin in the Heart

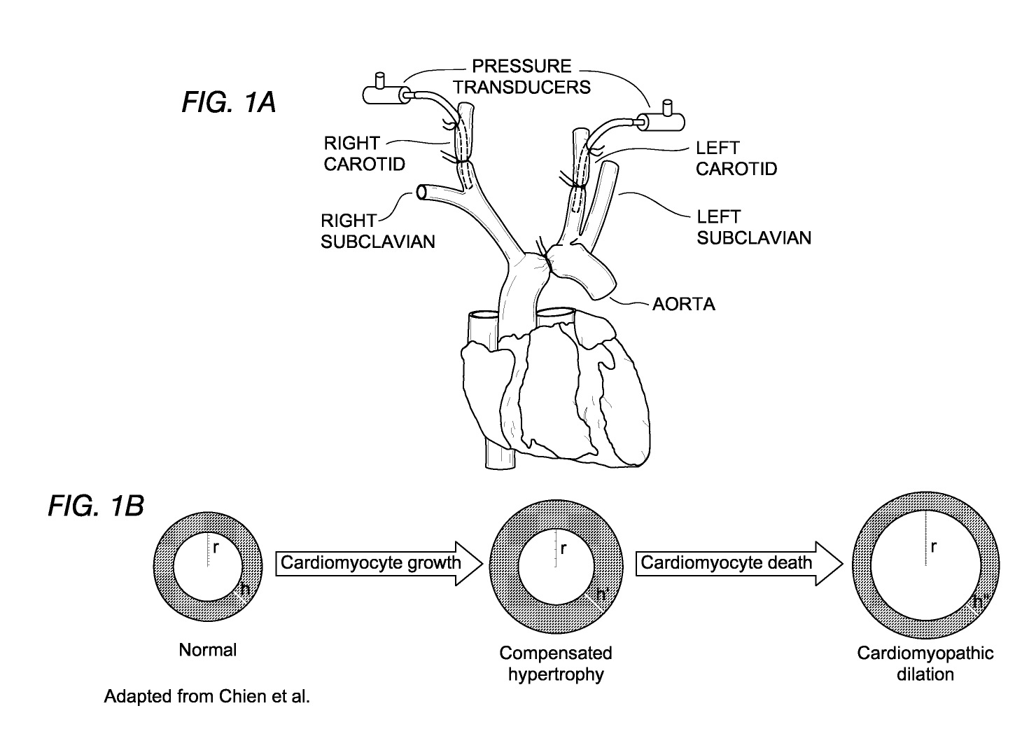

(A) T-cadherin and adiponectin co-localize on cardiomyocytes. Immunostaining of wild type, T-cadherin and adiponectin null hearts using T-cadherin and adiponectin antibodies revealed strong punctuate membrane staining for both antigens in the wild type mice. Adiponectin signals were abolished in the T-cadherin null condition, and T-cadherin signals were notably reduced in the adiponectin null heart. Supporting cardiomyocyte expression, longitudinal cardiomyocyte sections show rows of T-cadherin positive puncta co-localizing with APN on the sarcolemma. Bars, 5 μm. (B) Expression levels of T-cadherin mRNA in the myocardium of wild type and adiponectin null mice were similar. T-cadherin mRNA was detected at comparable levels in pure isolated cardiomyocytes (1° C.M). (C) Immunoblot of myocardial lysates of the indicated genotypes confirmed high T-cadherin expression in the wild type and low T-cadherin expression in adiponectin null samples. Adiponectin was abundant in wild type hearts, but below detection levels in T-cadherin null heart lysates. T-cadherin was detected as an approximately 130 kD pro-protein and a proteolytically cleaved approximately 105 kD doublet. Adiponectin appeared as a single 30 kD band under denaturing conditions. (D) Adiponectin ELISA of serum from wild type, T-cadherin null and adiponectin null mice. Serum adiponectin concentration was significantly increased in T-cadherin null mice (19.4±1.63 μg/mL) compared to wild type controls (5.6±0.13 μg/mL, n=4 per group, P<0.0001). Adiponectin was not detected in plasma from adiponectin null mice, confirming the specificity of the ELISA. Results are presented as mean±s.e.m.

FIG. 3. Serum Adiponectin in Wild Type, T-cadherin and Adiponectin Null Mice

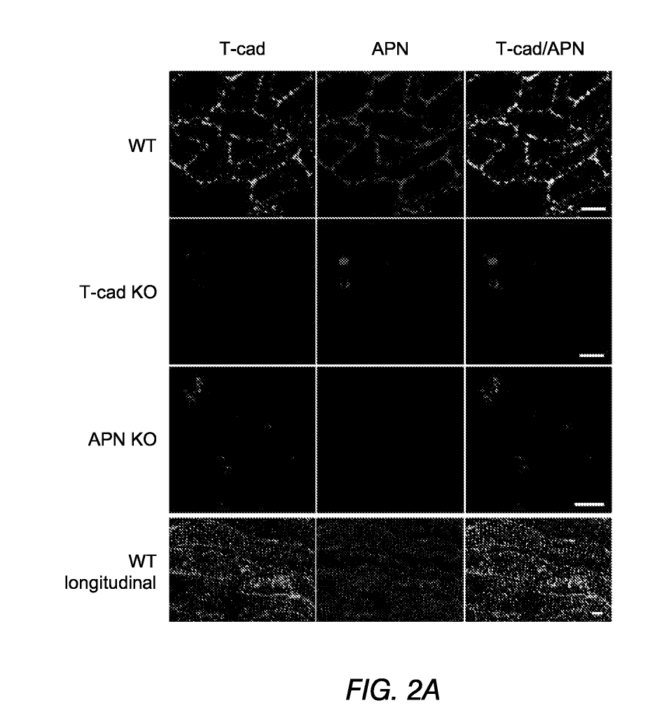

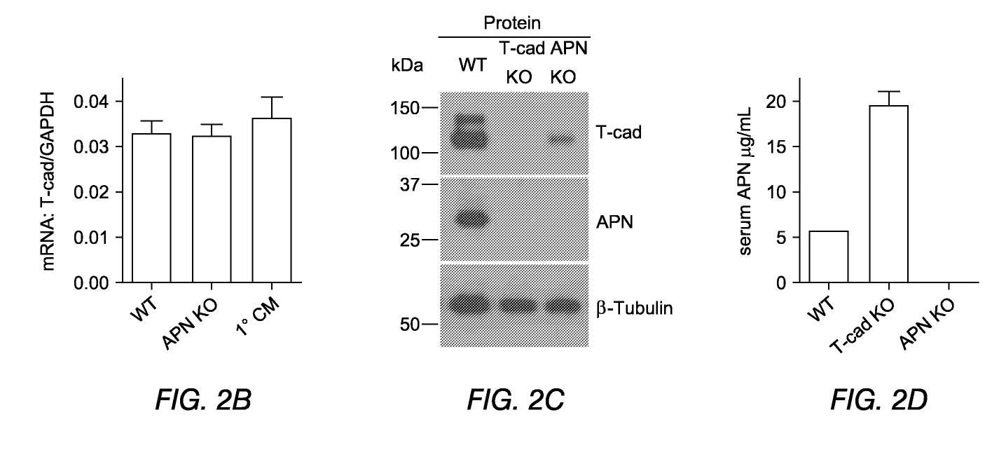

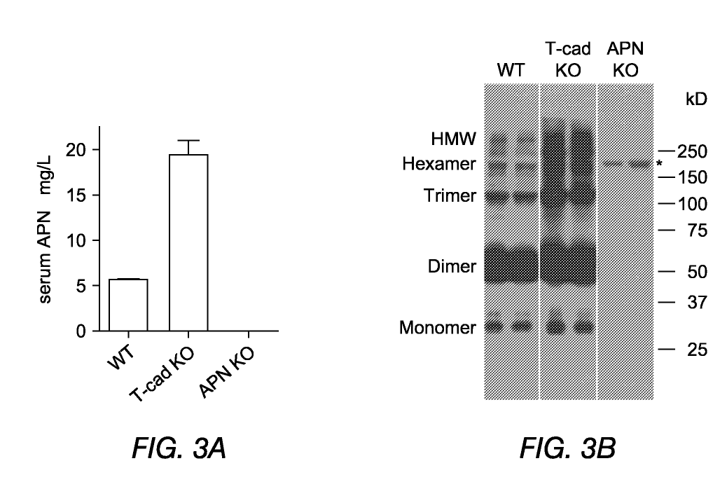

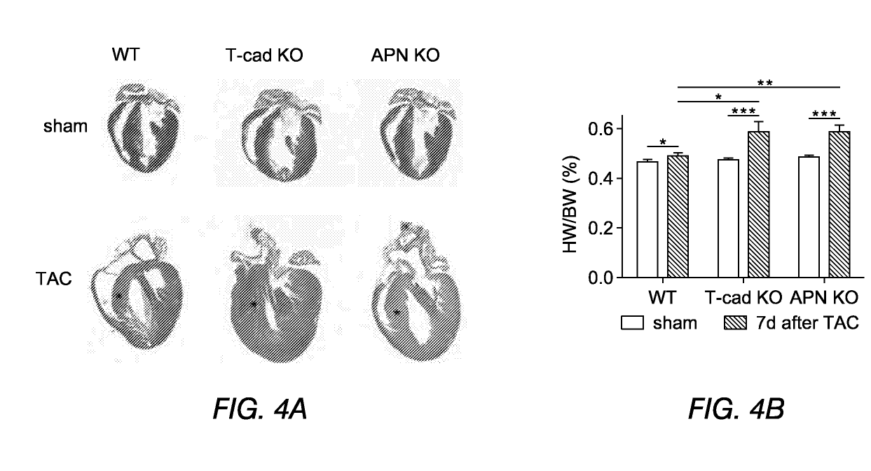

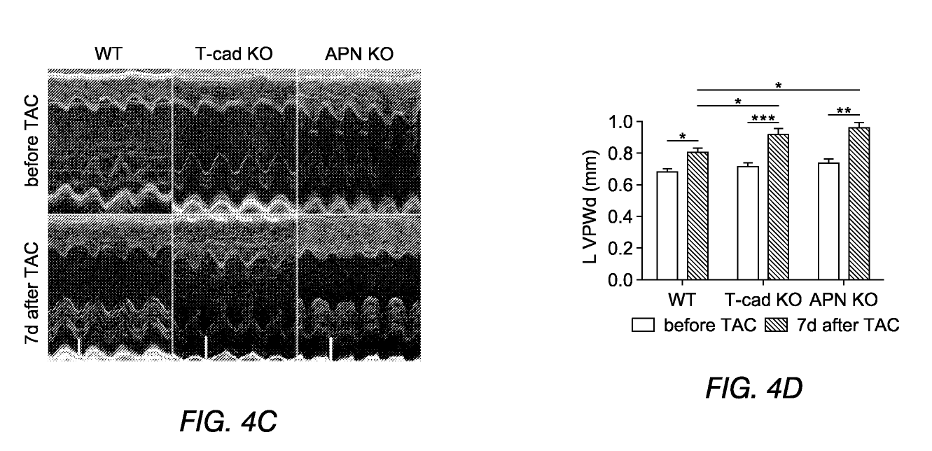

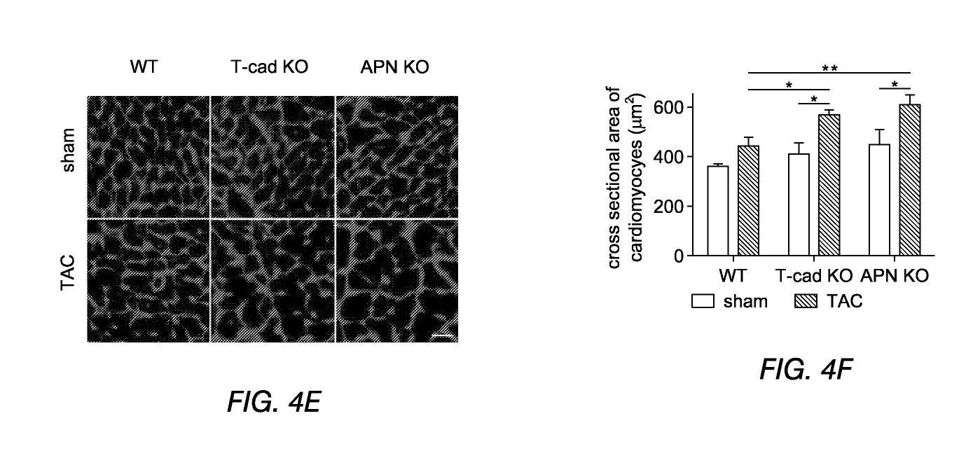

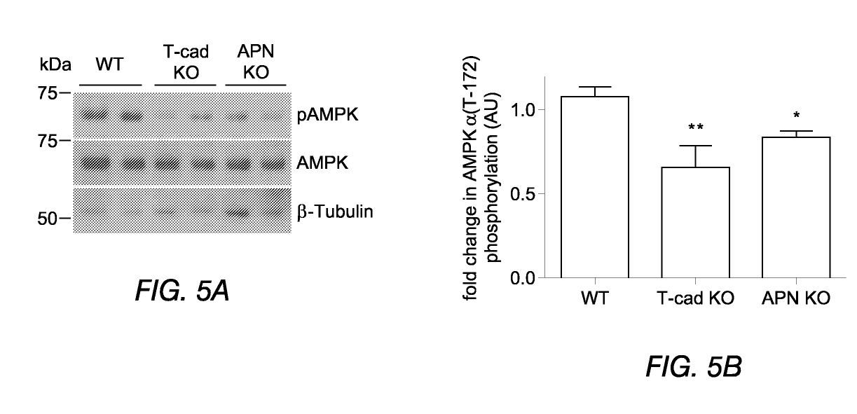

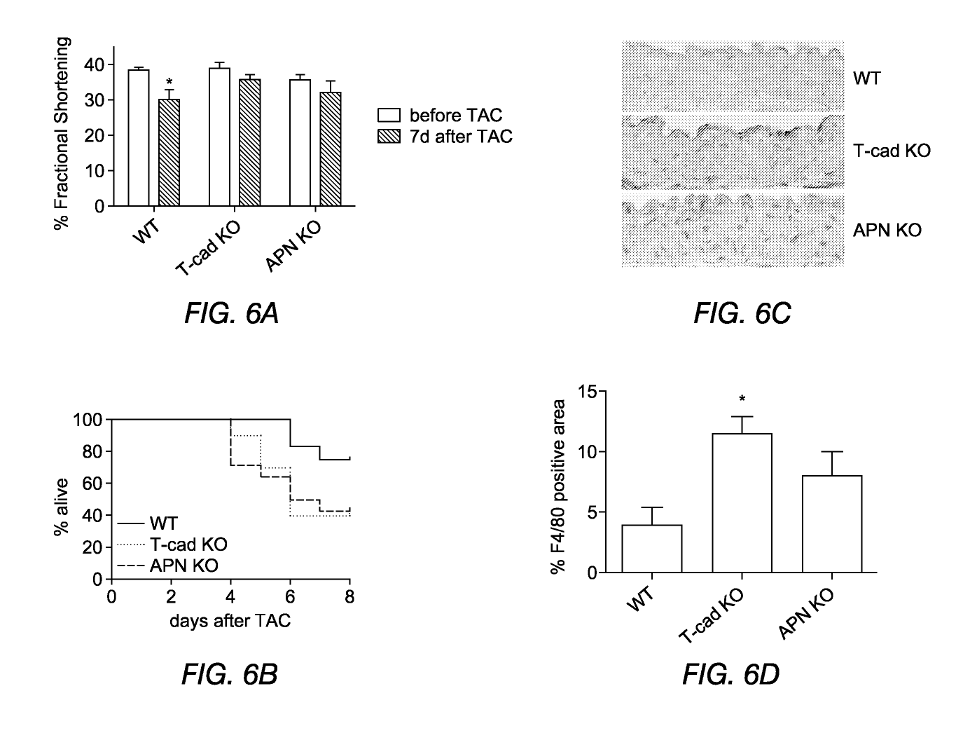

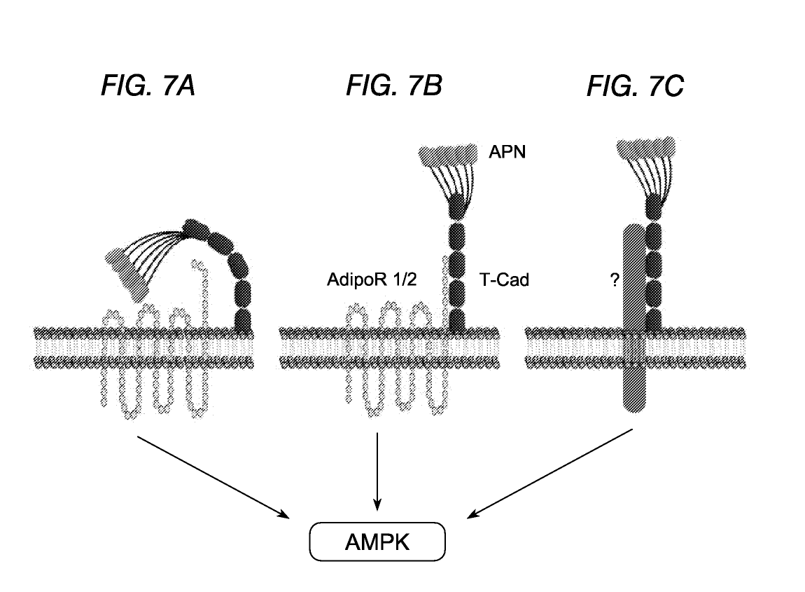

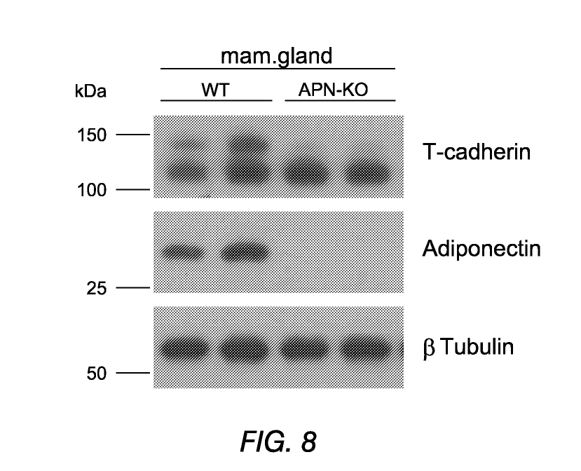

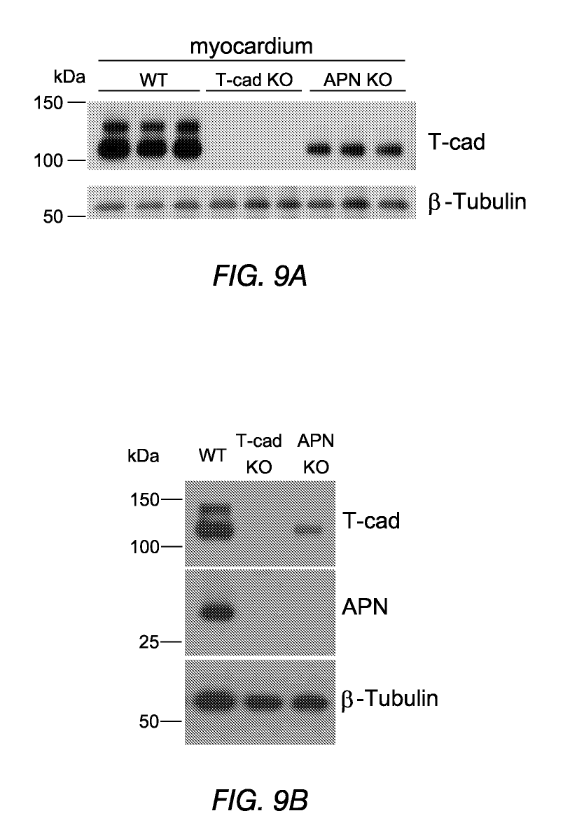

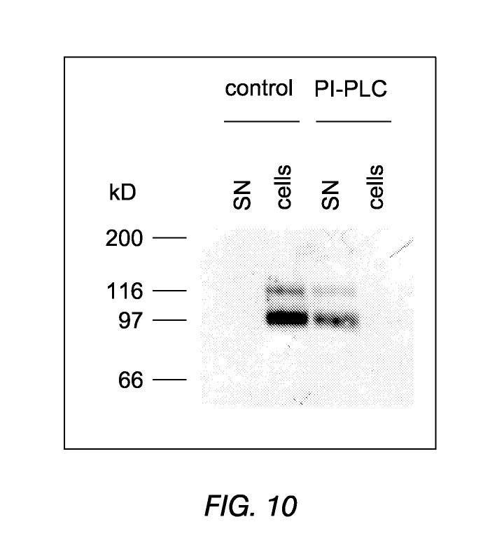

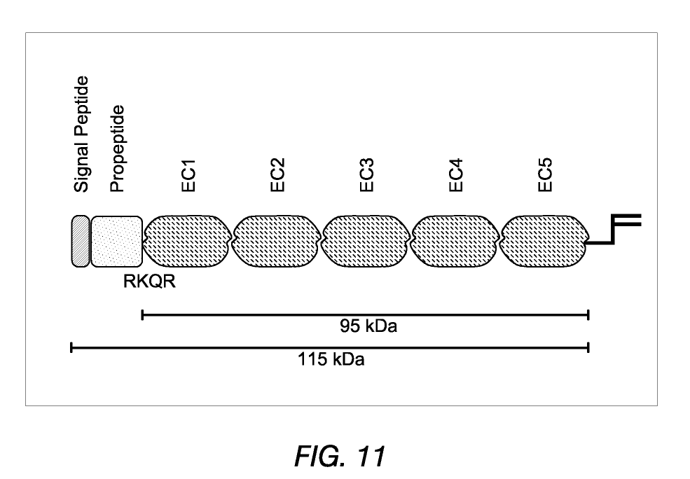

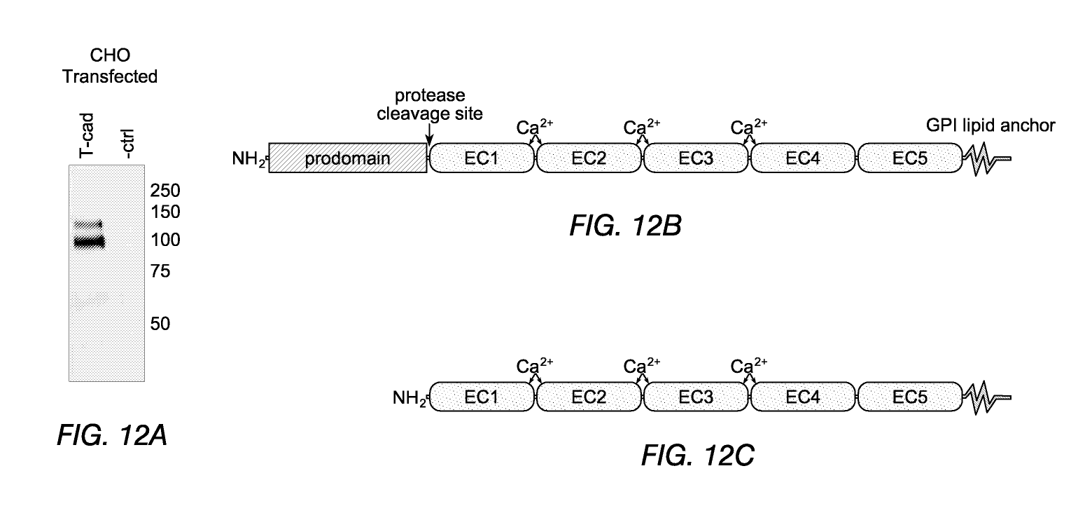

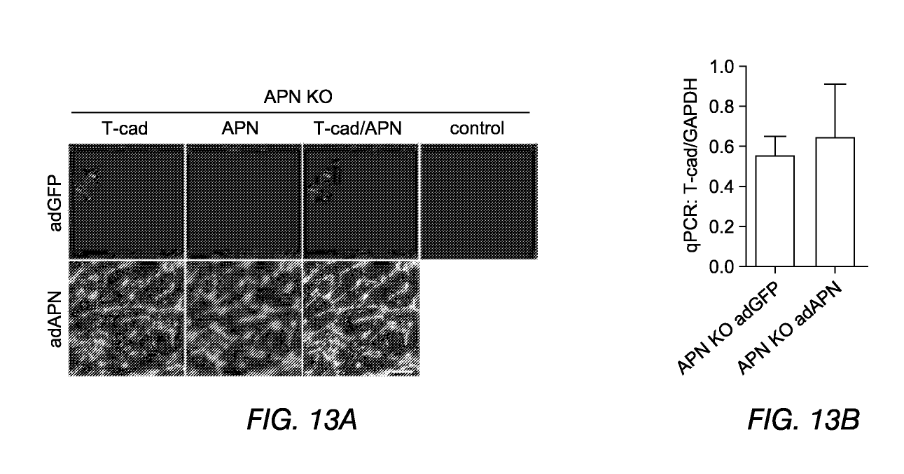

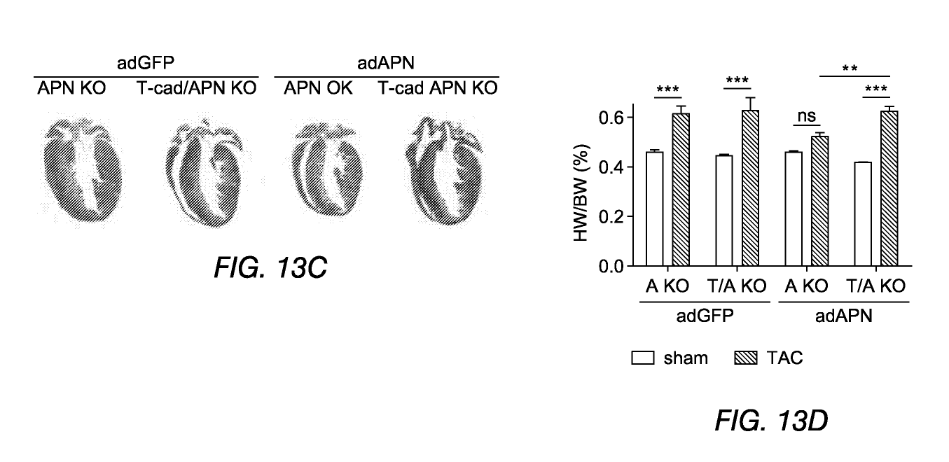

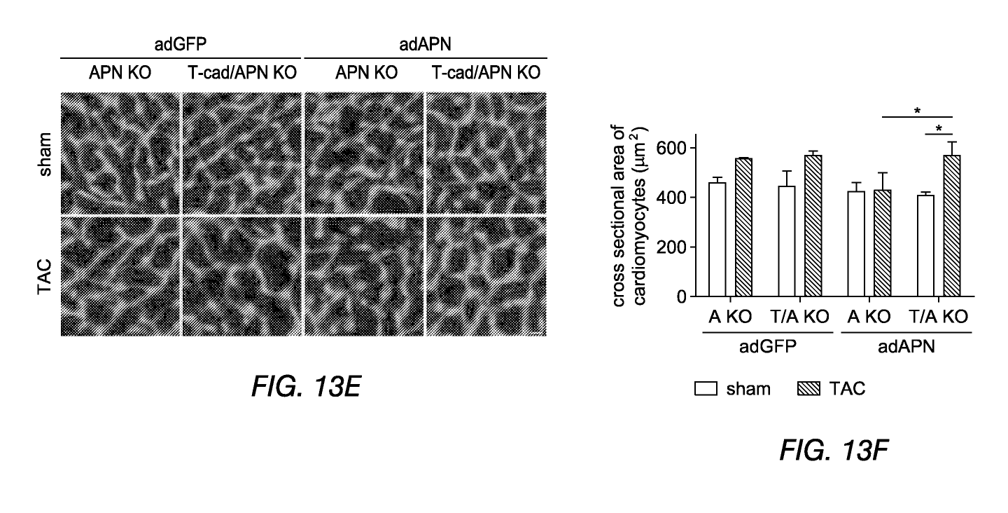

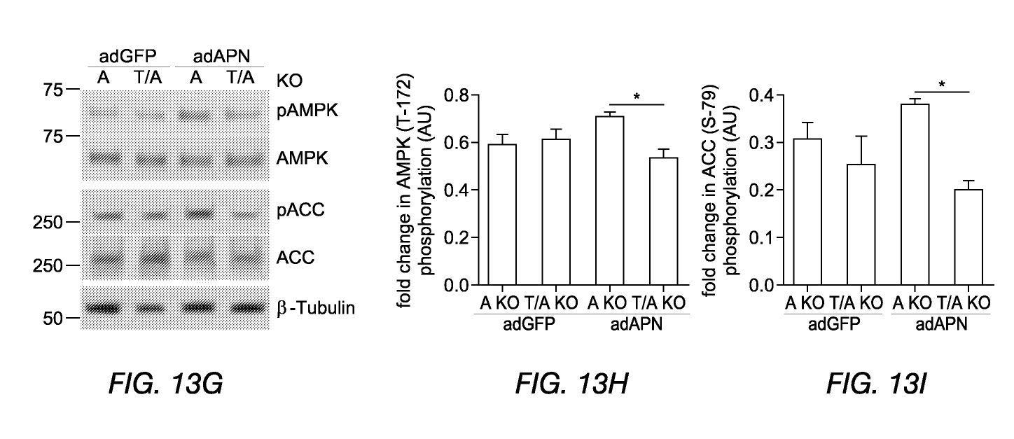

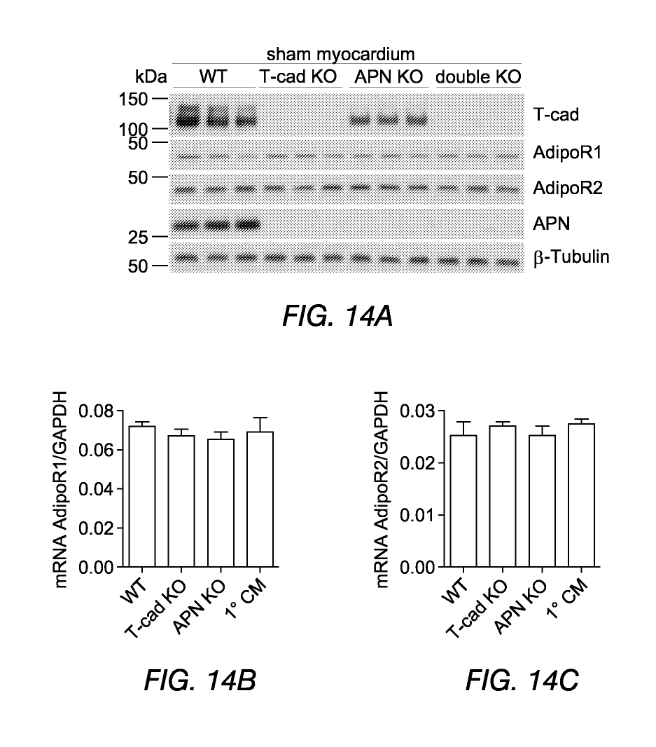

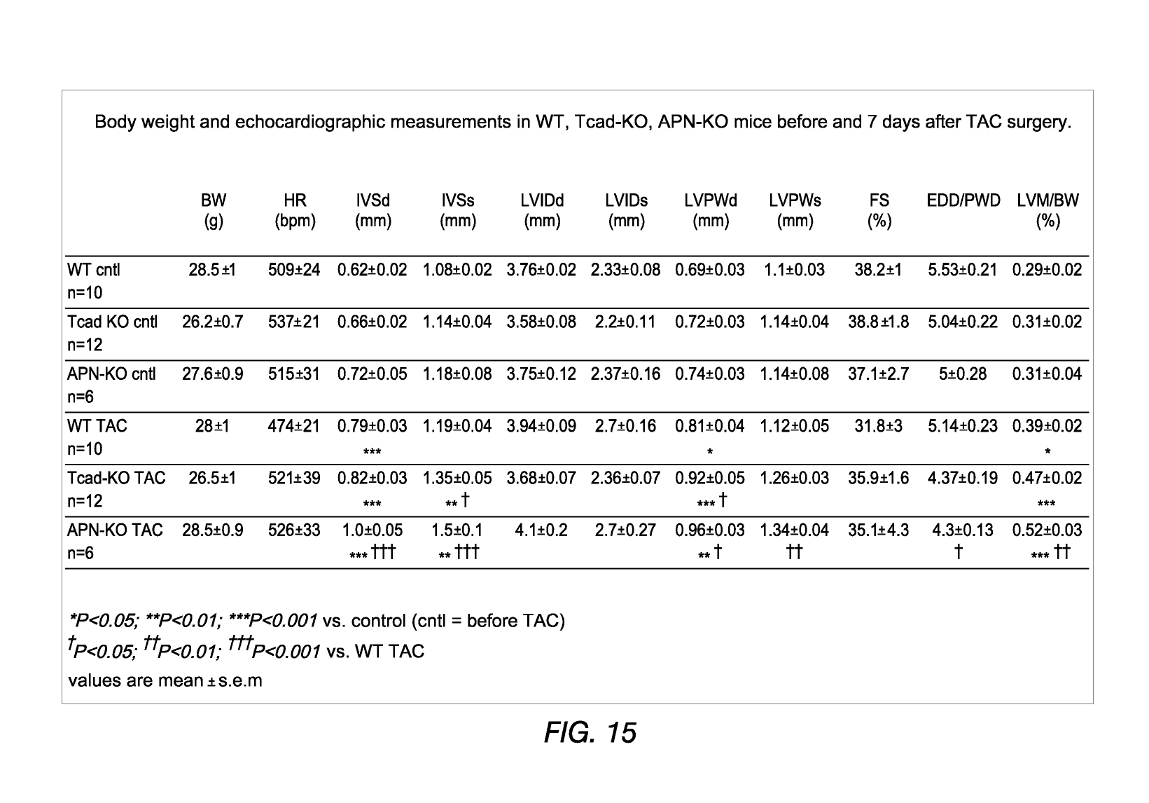

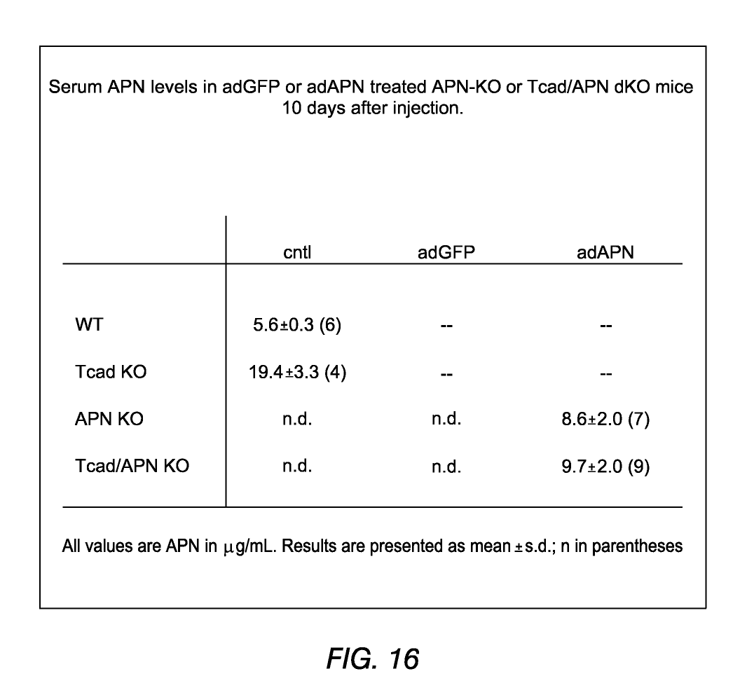

(A) See FIG. 2B above. (B) Immunoblot with plasma from wild type, T-cadherin and adiponectin null mice. 0.5 μL serum was loaded per lane and the cropped images are from the same blot under identical conditions. The symbol “*” denotes a nonspecific signal in the adiponectin null lanes and confirms loading of the lanes.

FIG. 4. Increased Cardiac Hypertrophy in T-cadherin Null and Adiponectin Null Mice after TAC

(A) Representative images of trichrome stained wild type, T-cadherin null and adiponectin null hearts seven days after sham or TAC surgery. Increases in the left ventricular walls in T-cadherin and adiponectin null mice versus wild type mice after TAC were observed (*). Bar, 2 mm. (B) Heart weight to body weight ratios were significantly increased in T-cadherin and adiponectin null mice after TAC (wild type, n=8; T-cadherin null, n=7; adiponectin null, n=11; *P<0.05, **P<0.01, ***P<0.001; one-way ANOVA). (C) M-mode echocardiograms from wild type mice, T-cadherin null and adiponectin null mice seven days after TAC surgery. M-mode echocardiograms display the cardiac wall thickness as a function of time and thus display systolic contractions. White bars indicate increased diastolic left ventricular wall thickness in the mutant mice after TAC. (D) Quantification of diastolic left ventricular posterior wall thickness (LVPWd) before and seven days after TAC surgery reveals significant increase in heart size in the wild type and exaggerated cardiac hypertrophy in T-cadherin null and adiponectin null mice (WT, n=10; T-cadherin null, n=14; adiponectin null, n=5; *P<0.05; **P<0.01; ***P<0.001; one-way ANOVA). (E) Fluorescent wheat germ agglutinin stained cell surfaces in hearts from wild type, T-cadherin null and adiponectin null mice seven days after sham or TAC surgery. An increased myocyte cross sectional area in T-cadherin null and adiponectin null mice after TAC was observed. Bar, 10 μm. (F) Quantification of myocyte cross sectional areas in (E) reveals a non-significant increase in cross sectional area in the wild type after TAC, and statistically significant myofiber enlargement in T-cadherin null and adiponectin null hearts (sham, n=3 per group; TAC, n=4 per group; *P<0.05, **P<0.01; one-way ANOVA).

FIG. 5. Decreased AMPK Phosphorylation in T-cadherin and Adiponectin Null Hearts after TAC

(A) Representative immunoblots from wild type, T-cadherin null and adiponectin null myocardial lysates seven days after TAC using phospho-specific (Thr-172) and total anti-AMPK antibodies with reference to 13-tubulin. AMPK phosphorylation was significantly reduced in both mutants. (B) Quantification of the immunoblots from (A) (wild type, n=5; T-cadherin null, n=4; adiponectin null, n=5; *P<0.05, **P<0.01; one-way ANOVA).

FIG. 6. Retained Function and Reduced Survival Coincided with Increased Vascular Inflammation in T-cadherin and Adiponectin Null Animals

(A) Fractional shortening (i.e., a measure of cardiac function) was analyzed using echocardiographic measurements. The wild type mouse displayed a significant reduction after TAC, while both mutant mice displayed only a slight nonsignificant reduction of cardiac function (*P<0.05; one-way ANOVA). (B) Survival of wild type, T-cadherin and adiponectin null animals after TAC. Survival of T-cadherin null and adiponectin null animals was significantly reduced compared to wild type controls (WT, n=12; T-cadherin null, n=10; adiponectin null, n=14; *P<0.05, Gehan-Breslow-Wilcoxon test shows statistical significance). (C) Macrophage-specific F4/80 immunostaining of sections from paraffin embedded normal wild type, T-cadherin null and adiponectin null aortas. (D) Quantification of F4/80 signal in the tunica media revealed a significant increase in macrophage invasion in T-cadherin null and adiponectin null aortas (WT, n=3; T-cadherin null, n=4; adiponectin null, n=2; *P<0.05; one-way ANOVA).

FIG. 7. Potential Models for Adiponectin Receptor Mechanism

(A) T-cadherin may solely serve in presenting adiponectin to Adipo1/2. (B) Adiponectin may directly interact with Adipo1/2, where the combined engagement of T-cadherin and AdipoR1/2 with adiponectin lead to signal transduction. (C) Another unidentified signaling protein may interact with T-cadherin and/or adiponectin, spanning the membrane to elicit intracellular signaling.

FIG. 8. Adiponectin Binds to the Intact Pro-Peptide Form of T-cadherin in the Mammary Gland

Western blot of T-cadherin in the mammary gland. In wild type mice, T-cadherin is expressed in the mammary gland in both the pro-peptide and mature forms (i.e., bands at about 120 kD and about 100 kD). In adiponectin null mice, T-cadherin is expressed in the mammary gland, but only in the mature form (i.e., a band at about 100 kD)—the pro-peptide form is missing. Equal loading is indicated in the control β-tubulin lane.

FIG. 9. The Intact Pro-Peptide Form of T-cadherin is Involved in the Association of Adiponectin with the Heart

Western blots of T-cadherin in the heart. (A) In wild-type mice, T-cadherin is expressed in the heart in both the pro-peptide and mature forms (i.e., bands at about 120 kD and about 100 kD). In adiponectin null mice, T-cadherin is expressed in the heart, but only in the mature form (i.e., a band at about 100 kD)—the pro-peptide form is missing. Equal loading is indicated in the control β-tubulin lane. (B) The experiment as shown in (A) with additional data indicating that adiponectin is not expressed in adiponectin null or T-cadherin null mice.

FIG. 10. T-cadherin Precursor is Expressed on the Cell Surface

Cleavage of the glycosylphosphatidyl inositol (GPI) anchor with phosphatidyinositol-specific phospholipase C (PI-PLC). Both the precursor and the mature forms of T-cadherin are cleaved by PI-PLC (which cleaves the protein off the membrane at the GPI anchor) and released into the culture supernatant (SN). This indicates that, unlike classical cadherins, the precursor form T-cadherin is expressed on the cell surface. The mature form of T-cadherin is also expressed on the cell surface.

FIG. 11. Pro-peptide Form of T-cadherin

The pro-peptide form of T-cadherin is detected as a molecular weight band of about 115-120 kD on Western blots (although this range can be greater due, e.g., to glycosylation differences between tissues or the type of gel used). This pro-peptide form is cleaved at the RKQR site by a pro-protein convertase to yield mature T-cadherin, which is detected as a molecular weight band of about 95-100 kD on Western blots (although this range can also be greater due, e.g., to glycosylation differences between tissues or the type of gel used). Thus, T-cadherin appears as a doublet of bands at about 120 kD and about 100 kD on Western blots (17).

FIG. 12. Pro-peptide and Mature Forms of T-cadherin

(A) Precursor and mature forms of T-cadherin in CHO transfected cells. (B, C) Depiction of the precursor and form and mature of T-cadherin. T-cadherin is expressed in the precursor form (about 120 kD—molecule with prodomain followed by EC1-EC5) and the mature form (about 100 kD—molecule with EC1-EC5), and is linked to the plasma membrane through a GPI anchor.

FIG. 13. Reversion of Exaggerated Cardiac Hypertrophy Phenotype in APN-KO Depends on T-cadherin

(A) T-cadherin protein expression was induced by adenovirus-mediated adiponectin expression in adiponectin null hearts (2×10⁸ p.f.u./mouse). Immunostaining with T-cadherin and adiponectin antibodies revealed low T-cadherin expression in GFP-expressing control adenovirus (adGFP) treated mice (control staining confirmed the absence of GFP signals in the heart). T-cadherin protein expression by cardiomyocytes was restored by adenovirus-expressed recombinant adiponectin (adAPN) treatment. Adenovirus-expressed adiponectin co-localized with T-cadherin in the heart. (B) Cardiac T-cadherin mRNA levels measured by qPCR remained unchanged in adGFP-(n=5) or adAPN-treated (n=3) adiponectin null mice. (C) Representative trichrome-stained heart sections seven days after TAC revealed that adenovirus mediated adiponectin expression leads to reduced hypertrophic responses only in adiponectin null, but not in T-cadherin/adiponectin double knockout (“T-cad/APN KO”) mice. Control adGFP-treated mice showed no reduction in cardiac growth. (D) Ratios of heart weights-to-body weights (HW/BW) indicate exaggerated hypertrophy in control adGFP-treated adiponectin null (“A KO”) and T-cadherin/adiponectin double KO (“T/A KO”) mice after TAC. adAPN-treatment blunted cardiac hypertrophy selectively in the adiponectin null condition, but not in the absence of T-cadherin in T-cadherin/adiponectin double KO mice. (adGFP, n=3 each; adAPN, n=4 each; **P<0.01; ***P<0.001; one-way ANOVA). (E) Representative images of wheat-germ agglutinin stained cell surfaces confirm the selective suppression of hypertrophy after TAC in myocytes of the free wall of the left ventricle in adAPN-treated mice. This was quantified in (F). There was a significant reduction of cardiomyocyte cross-sectional area in adiponectin null mice, but not in T-cadherin/adiponectin double KO mice when treated with adAPN. (adGFP, n=3 each; adAPN, n=4 each; *P<0.05; one-way ANOVA). (G) Adenoviral-mediated adioponectin expression induced cardiac AMPK and ACC signaling only in adiponectin null mice, but not in T-cadherin/adiponectin double KO animals. Representative immunoblots seven days after TAC are shown. These differences were statistically significant (H, I) (adGFP, n=3 each; adAPN, n=4 each; *P<0.05; one-way ANOVA).

FIG. 14. AdipoR1/2 in the Heart

(A) Immunoblotting of myocardial lysates revealed high T-cadherin expression in the wild type and low T-cadherin expression in adiponectin null samples. AdipoR1 and AdipoR2 were detected in wild type, T-cadherin null, adiponectin null, and T-cadherin/adiponectin double KO without genotype-dependent changes. (B, C) Expression levels of AdipoR1 and AdipoR2 mRNA were unchanged in hearts of wild type, T-cadherin null and adiponectin null mice. Preparations of pure isolated wild type cardiomyocytes (1° C.M) confirmed expression levels of the respective genes.

FIG. 15. Body Weight and Echocardiographic Measurements

Body weight and echocardiographic measurements in wild type, T-cadherin null and adiponectin null mice before and seven days after TAC surgery.

FIG. 16. Serum Adiponectin Levels

Serum adiponectin levels in adGFP- or adAPN-treated adiponectin null or T-cadherin/adiponectin double KO (“Tcad/APN KO”) mice ten days after injection.

### DETAILED DESCRIPTION

Embodiments relate to the discovery that T-cadherin and adiponectin null mice display similar phenotypes, that adiponectin is displaced from target tissues in T-cadherin null animals, and that T-cadherin is required for adiponectin binding to cells. These data provide strong evidence of a functional interaction between T-cadherin and adiponectin, and support the embodiments described herein.

As described herein, adiponectin co-localizes with T-cadherin on cardiomyocyte membranes. This interaction was abolished in T-cadherin null mice. To test the hypothesis that T-cadherin is functionally involved in adiponectin-mediated cardioprotection, T-cadherin and adiponectin null mice were subjected to pressure overload in order to induce cardiac hypertrophy. Follow-up analyses were performed after seven days. Compared to wild type controls, both mutants exhibit increased hypertrophy as measured by heart weight to body weight ratio, left ventricular wall thickness and cross-sectional area of single cardiomyocytes. Phosphorylation of Thr-172 of the α-subunit of AMPK was reduced in both mutant mice, suggesting that T-cadherin is essential for adiponectin-dependent AMPK activation. Mortality was significantly increased in T-cadherin and adiponectin null mice, and the animals exhibited aortic rupture three to seven days after TAC. Analysis of aortas revealed that macrophage infiltration into the aortic wall was significantly increased in T-cadherin and adiponectin null mice. Together, these data suggest that, as an essential component of adiponectin signaling, T-cadherin protects from cardiac hypertrophy and aortic rupture.

All physiological adiponectin isoforms interact with T-cadherin, and T-cadherin docks adiponectin to cells in its target tissues. Without T-cadherin, adiponectin is undetectable at cell surfaces and is coincidentally found at supra-physiological levels in the blood stream.

Contrary to the wide-held belief that adiponectin protects from breast cancer, adiponectin provided a pro-angiogenic contribution to mammary tumorigenesis. An adiponectin null mouse model for breast cancer revealed that tumor angiogenesis is limited in the absence of adiponectin. Decreased vascularization delays tumor onset and progression, and increased survival, but does not provide a long-term effect. The rate of pulmonary metastasis in the adiponectin null condition was significantly increased. This phenotype is of striking similarity to the tumor phenotype in the T-cadherin null mouse using a breast cancer model as previously reported (18). In T-cadherin null mice, tumors were similarly restricted by limited vascular supply. Transplantation studies have shown that this is due to lack of T-cadherin in the tumor microenvironment, providing evidence of a non-cell-autonomous effect. The parallel phenotypes provide strong evidence of a concerted function of the two proteins in the vasculature. In contrast, a recent study using the MMTV-PyV-mT model in mice heterozygous for the adiponectin gene mutation found a cancer-protective role of adiponectin (19). However, interpretation of these data must be made with caution, as the researchers report embryonic lethality of the adiponectin null mouse, which is in contradiction to the adiponectin null mouse described herein.

Adiponectin has a known role in cardioprotection, and T-cadherin is abundantly expressed in the heart. These observations suggest a role for T-cadherin in mediating the cardiac functions of adiponectin. Pressure overload-induced cardiac remodeling is enhanced in both T-cadherin and adiponectin null animals. The increase in heart size is accompanied by reduced AMPK phosphorylation, indicating that T-cadherin and adiponectin cooperate in activating this pathway. Both adiponectin and T-cadherin null mice have at least mildly inflamed aortas that are prone to rupture.

Adiponectin activates AMPK in the heart and thus protects it against the deleterious consequences of cardiac hypertrophy. It has been shown that cardiac hypertrophy in adiponectin null mice can be reduced using an adiponectin-encoding adenovirus, thus rescuing the phenotype. If T-cadherin were indeed a required ligand-binding receptor of adiponectin, this rescue would be impossible in mice lacking both T-cadherin and adiponectin.

T-cadherin null mice have surprisingly high serum concentrations of adiponectin. However, these mice do not show phenotypes that would be consistent with enhanced bioactivity of adiponectin. Instead, T-cadherin null mice phenocopy adiponectin null mice with respect to angiogenesis, cardiac hypertrophy and vascular inflammation. Thus, these tissues appear entirely insensitive to adiponectin in the T-cadherin null condition, despite elevated adiponectin serum levels. Thus, adiponectin abundance in the circulation has little relevance as an isolated parameter, as adiponectin sensitivity of tissues is governed by T-cadherin. This role of T-cadherin is indirect evidence of a strict requirement of T-cadherin for adiponectin's functions.

Receptors typically serve the dual function of binding a ligand from the environment and subsequently transducing a signal into the cell's interior. In the case of adiponectin, distinct molecules may serve these functions. T-cadherin appears be the ligand binding-receptor for adiponectin, as genetic deletion of T-cadherin abolishes adiponectin binding to cellular surfaces.

T-cadherin may dock adiponectin and transduce its signal into the cell's interior in one of at least three ways. First, T-cadherin might serve as a ligand binding receptor that tethers adiponectin to cell surfaces and thus functions in presenting adiponectin to signal transducing transmembrane receptors. AdipoR1/2 might serve this role as the signaling component of a hypothetical multiprotein receptor complex. AdipoR1/2 are expressed in cardiomyocytes (FIG. 14), and experimental evidence suggests that they can activate the AMPK pathway upon stimulation with adiponectin (20). AdipoR1/2 respond specifically to the globular domain of adiponectin. Adiponectin is strongly reduced in T-cadherin null myocardium, suggesting that cardiac AdipoR1/2 are disengaged. Reduced AMPK phosphorylation in the T-cadherin and adiponectin mutant animals is consistent with this finding. These data raise the possibility that T-cadherin might present adiponectin to AdipoR1/2, which then transduce this signal to elicit intracellular responses (FIG. 7A).

Second, adiponectin may directly interact with AdipoR1/2, and combined engagement of T-cadherin and AdipoR1/2 with adiponectin would lead to signal transduction (FIG. 7B). It is further possible that AdipoR1/2 are removed from the membrane in the absence of T-cadherin. Examples from the literature show that other GPI linked molecules play important roles in intracellular events. For example, contactin (a neuronal GPI-linked molecule that is highly enriched in the nodes of Ranvier) associates with the transmembrane molecule Caspr (contactin associated protein), and thus links to the cytoskeleton (21). Another example comes from the Ephrin/Eph receptor field. Ephrin-As are GPI-linked membrane bound ligands of Eph receptors that can engage in “reverse” signaling. This depends on their association with p75 neurotrophin receptor (NTR) (22), which initiates signaling through the src-related protein-tyrosine kinase Fyn.

Third, an unidentified signaling protein may interact with T-cadherin and/or adiponectin, spanning the membrane to elicit intracellular signaling (FIG. 7C). This pathway would be parallel to AdipoR1/2-dependent adiponectin signaling, with the two mechanisms converging on AMPK. However, AMPK activation is linked to the cardioprotective effects of adiponectin—independent of its upstream activators (23)

The symmetrical structure of the physiological higher molecular weight forms of adiponectin suggests a function in clustering membrane proteins. Receptor clustering is a common mechanism for receptor activation, e.g., in receptor tyrosine kinases. Adiponectin may have a role in organizing large protein complexes at the cell membrane that include T-cadherin and signal transducing transmembrane proteins, resulting in the formation of signaling platforms. In some embodiments, these ideas are further tested using in vitro and in vivo systems.

GPI-linked proteins such as T-cadherin partition to lipid rafts, which are specialized membrane microdomains with elevated cholesterol and sphingolipid content (24). Lipid rafts are highly active membrane sites that are implicated in endocytosis, virus budding, apical/basolateral sorting, and serve as signaling platforms. Lipid rafts are sites for G-protein coupled receptor signaling (25). There is evidence for ion channel localization in lipid rafts (26, 27), and lipid rafts can be enriched with Src and EGFR kinases (28). Thus, recruitment of T-cadherin to lipid rafts may facilitate T-cadherin dependent adiponectin signaling. However, the distribution of adiponectin receptors within cellular membranes is unknown. In some embodiments, the role of these signaling microdomains in T-cadherin functions is further investigated.

T-cadherin interacts with adiponectin in a physiologically relevant way, and T-cadherin is involved in adiponectin's effects on its target tissues. In some embodiments, further studies are conducted to gain new insight into the physiological role of the T-cadherin/adiponectin interaction. For example, in some embodiments, genetically modified mice are studied to clarify the role of T-cadherin in the various components of the tumor microenvironment, and to distinguish vascular and cardiac contributions of the cardiovascular phenotypes. In some embodiments, the genetically modified mice carry tissue-specific null mutations in the T-cadherin locus.

In some embodiments, molecules that directly associate with T-cadherin to mediate its function are identified (e.g., using a proteomics approach). The kinases linking adiponectin reception to AMPK are identified. In addition, other T-cadherin-dependent downstream targets of adiponectin may be identified through this approach. In one embodiment, adiponectin target tissues are assessed for differences regarding signal transduction. In some embodiments, modulation of the sensitivity of adiponectin is undertaken in order to reverse deleterious effects of hypoadiponectinemia.

The embodiments described herein have potential biotechnological, diagnostic, and therapeutic uses. For example, the proteins and binding partners described herein can be incorporated into pharmaceuticals and used to confer cardioprotective effects in a mammal.

The proteins described herein can encompass adiponectin and/or T-cadherin and proteins that are functionally equivalent to adiponectin and/or T-cadherin, including polypeptides, fragments, and chemicals that resemble these molecules (e.g., peptidomimetics, modified proteins, and derivatives or variants of adiponectin and/or T-cadherin). For example, embodiments can include mutant adiponectins and/or T-cadherins with nonconservative amino acid replacements, such as mutants that result in gain or loss of function. Further, the embodiments described herein can include adiponectin and/or T-cadherin-like hybrids having one or more domains deleted or combined in a novel fashion. Adiponectin and/or T-cadherin can also include multimerized domains, synthetic domains, or domains from other signal transduction proteins. Embodiments can also include polypeptides that have homology to adiponectin and/or T-cadherin.

In some embodiments, isolated or purified proteins can be used to generate antibodies and other tools useful for identifying agents that interact with adiponectin and/or T-cadherin. These antibodies can encompass polyclonal, monoclonal, chimeric, single chain, Fab fragments, and fragments produced by a Fab expression library. Antibodies that recognize adiponectin and/or T-cadherin have many uses, including biotechnological applications, therapeutic/prophylactic applications, and diagnostic applications.

The adiponectin and T-cadherin assays described herein can be used to test the functionality of molecules and identify binding partners that interact with adiponectin and/or T-cadherin. For example, functional assays can involve the use of adiponectin and/or T-cadherin disposed on a support (e.g., a resin, bead, lipid vesicle or cell membrane), in which the adiponectin and/or T-cadherin is contacted with a candidate binding partner and an association of the binding partner is determined. This binding can be determined directly (e.g., using a labeled binding partner) or indirectly (e.g., using a labeled antibody directed to the binding partner). The molecules described herein can be labeled by any approach known to one of skill in the art (e.g., radioactivity or fluorescence). In some embodiments, samples with labeled proteins can be applied to an array under conditions that permit binding—allowing for detection of the presence, concentration, and/or expression level of a protein. Successful binding partners can be further analyzed for their effect on adiponectin and/or T-cadherin function using cell-based assays. For example, these cell-based assays can be used to rapidly identify binding partners that interact with an adiponectin and/or T-cadherin, and thereby modulate signal transduction.

Candidate binding partners for adiponectin and T-cadherin can be obtained from chemical or peptide libraries. For example, monoclonal antibodies that bind to an adiponectin and/or T-cadherin can be created, and the nucleic acids encoding the VH and VL domains of the antibodies can be sequenced. These sequences can then be used to synthesize peptides that bind to the adiponectin and/or T-cadherin. Further, peptidomimetics corresponding to these sequences can be created and used as candidate binding partners.

The transgenic animals described herein can be engineered to express wild-type or mutant adiponectin and/or T-cadherin, fragments of adiponectin and/or T-cadherin, or adiponectin and/or T-cadherin-like hybrids. These transgenic animals may exhibit point mutations or complete knockouts of one or more existing genes. Any technique known in the art can be used to produce founder lines, replace existing adiponectin and/or T-cadherin genes, or generate knockouts. Embodiments also provide for transgenic animals that carry an adiponectin and/or T-cadherin transgene in all their cells, as well as animals that carry a transgene in some, but not all cells (e.g., tissue-specific mutations).

### EXAMPLES

### Example 1

### T-cadherin and Adiponectin Co-localize on Cardiomyocyte Surfaces

Wild type, T-cadherin null and adiponectin null hearts were immunostained to determine the location of T-cadherin and adiponectin. Heart sections were deparaffinized, incubated with Target Retrieval Solution (Dako, Denmark) and pretreated with 1% Saponin (Fluka, Buchs, Switzerland) in PBS with 1 mM EGTA. Autofluorescence was quenched with saturated Sudan Black (MP Biomedicals, Santa Ana, Calif.) solution in 70% ethanol and sections were blocked with Antibody Diluent (Dako, Denmark). Sections were then incubated with adiponectin (PA1-054, Affinity Bioreagents, Golden, Colo.) or T-cadherin antibodies (18). Alexa 488 and Alexa 594 fluorescent conjugates (MOLECULAR PROBES®, Invitrogen, Carlsbad, Calif.) were used to detect the respective primary antibodies. Negative controls were slides processed in parallel without primary antibody. For T-cadherin and adiponectin antibodies, tissues from knockout (“KO” or “null”) animals were used as additional controls.

Immunohistochemistry revealed that T-cadherin was strongly expressed on the surfaces of all cardiomyocytes, where it co-localized with adiponectin in wild type mice in cross-sections and on the sarcolemma in longitudinal views (FIG. 2A). In T-cadherin null hearts, no T-cadherin signal was detected—confirming the genetic disruption of the T-cadherin locus. Moreover, the cardiomyocyte surface staining for adiponectin was reduced to background levels, supporting the notion that T-cadherin sequesters adiponectin in the heart. Interestingly, T-cadherin's distribution in the heart was also changed in adiponectin null mice. The myocyte surface expression observed in the wild type was strongly reduced in the absence of adiponectin, suggesting that T-cadherin surface expression or stability in cardiomyocytes is influenced by its interaction with adiponectin.

### Example 2

### T-cadherin Expression and Adiponectin Association in the Heart

T-cadherin and adiponectin protein levels in the heart were confirmed by Western blotting, which further supported the interrelation between T-cadherin and adiponectin in the heart. In myocardial lysates, T-cadherin was detected as a pro-peptide-containing form of 120 kD and as a mature protein of 100 kD. T-cadherin was markedly reduced in adiponectin null heart tissue. Adiponectin was detected as a band of about 29 kD after SDS PAGE under reducing conditions, and was below detection level in T-cadherin null heart tissue (FIG. 2C).

Expression levels of the T-cadherin, AdipoR1 and AdipoR2 transcripts were measured using quantitative real-time PCR. Total RNA was extracted from cardiac tissue using Trizol (Invitrogen, Carlsbad, Calif.). Equal amounts were reverse transcribed using oligo (dT)₁₈ and random hexamer primers (Transcriptor First Strand cDNA Synthesis Kit, Roche, Pleasanton, Calif.). Real-time PCR analysis was performed with SYBR green using the Stratagene Mx3000p instrument. T-cadherin (5′-catcgaagctcaagatatgg-3′ (SEQ ID NO: 1); 5′-gatttccattgatgatggtg-3′ (SEQ ID NO:2)), AdipoR1 (5′-acacagagactggcaacatc-3′ (SEQ ID NO:3); 5′-gagcaatccctgaatagtcc-3′ (SEQ ID NO:4)), and AdipoR2 (5′-tggacacatctcctaggttg-3′ (SEQ ID NO:5); 5′-tagagaagagtcgggagacc-3′ (SEQ ID NO:6)) cDNAs were detected and normalized to GAPDH (5′-ccagtatgactccactcacg-3′ (SEQ ID NO:7); 5′-gactccacgacatactcagc-3′ (SEQ ID NO:8)).

T-cadherin mRNA was expressed in isolated neonatal primary cardiomyocytes. Both T-cadherin and adiponectin were abundant in wild type myocardial lysates. Consistent with the immunostaining results, adiponectin was not detected in T-cadherin null heart tissue, and vice versa, T-cadherin was markedly reduced in the adiponectin null myocardium T-cadherin mRNA levels in the heart were unchanged in adiponectin null mice compared to wild type mice, indicating that the reduction of T-cadherin protein in adiponectin null hearts occurs independently of transcriptional regulation. In fact, similar T-cadherin mRNA levels were found in wild type isolated primary cardiomyocytes (FIG. 2B).

### Example 3

### Cooperation of AdipoR1, AdipoR2, and T-cadherin

To investigate the potential cooperation of transmembrane receptors AdipoR1 and/or AdipoR2 with T-cadherin, the cardiac expression of these receptors was further explored. Immunoblotting was performed as described herein. Quantitative real-time PCR was performed as described in Example 2.

Immunoblotting of myocardial lysates revealed high levels of T-cadherin expression in the wild type and low levels of T-cadherin expression in adiponectin null samples. AdipoR1 and AdipoR2 were detected in wild type, T-cadherin null, adiponectin null, and Tcad/APN dKO without genotype-dependent changes. Expression levels of AdipoR1 and AdipoR2 mRNA were unchanged in hearts of wild type, T-cadherin null and adiponectin null mice. AdipoR1 and AdipoR2 mRNA and protein were detected in the myocardium with unchanged concentrations between the wild type, T-cadherin null and adiponectin null hearts (FIGS. 14A, 14B).

These data suggest that cardiac AdipoR1 or AdipoR2 activation may require T-cadherin, as adiponectin is absent from T-cadherin null hearts. T-cadherin's role as an adiponectin binding protein in the heart is further supported by its unique regulation by adiponectin serum concentrations. Together, these findings highlight T-cadherin's role in adiponectin functions and suggest that T-cadherin acts as a gateway receptor for enabling adiponectin functions in the heart.

### Example 4

### Adiponectin Oligomerization and Secretion by Adipose Tissue are Not Impaired in T-cadherin Null Mice

Adiponectin can exert some of its functions through AdipoR1/2, but the role of AdipoR1/2 in cardiac adiponectin function is unclear.

mRNA expression levels of adiponectin receptors AdipoR1 and AdipoR2 were measured. mRNA expression levels of adiponectin receptors AdipoR1 and AdipoR2 were similar between wild type, T-cadherin null and adiponectin null heart tissue, and comparable to wild type isolated primary cardiomyocytes (FIGS. 2B, 14).

The finding that adiponectin associates with cell surfaces in a T-cadherin dependent manner suggests that T-cadherin-positive myocyte surfaces bind adiponectin. Adiponectin concentrations and isoform distributions were tested to determine whether the serum of wild type and T-cadherin null mice differ. As shown in FIG. 2D, physiological total adiponectin levels were detected in wild type mice, and adiponectin was not detected in the adiponectin null condition. However, in T-cadherin null mice, the adiponectin serum concentration was increased at least four-fold compared to the wild type. Measurements of circulating adiponectin levels by ELISA revealed average concentrations of 5.6 μg/mL in wild type and 19.4 μg/mL in T-cadherin null mice (FIG. 2D). This marked increase in circulating adiponectin levels combined with loss of tissue-bound adiponectin in T-cadherin null mice suggests that T-cadherin sequesters adiponectin to the heart.

Non-reducing Western blots were run with plasma from wild type, T-cadherin null and adiponectin null mice. The relative composition and abundance of each of the adiponectin isoforms was unchanged between genotypes as determined by serum analysis (FIG. 3B). This composition analysis also confirmed the increase in total adiponectin amount in the T-cadherin null condition. Together, these data indicate that adiponectin oligomerization and secretion by adipose tissue are not impaired in T-cadherin null mice, and suggest a role for T-cadherin in making adiponectin available to target tissues.

### Example 5

### T-cadherin and Adiponectin Null Mice Show Increased Cardiac Hypertrophy During Pressure Overload

The strong co-dependence of cardiac T-cadherin expression and adiponectin association further raised the possibility that loss of T-cadherin renders the myocardium insensitive to adiponectin. To test this model, cardiac hypertrophy was analyzed during TAC-induced pressure overload in T-cadherin null mice. For reference, all experiments were carried out in parallel in wild type mice as negative controls and with adiponectin null mice for which the pressure-overload hypertrophic phenotype was previously established (8).

T-cadherin and adiponectin null mice display normal heart morphology and function under baseline conditions. Accordingly, hearts of sham operated wild type, T-cadherin null and adiponectin null mice were also similar in size (FIG. 4A). To test T-cadherin's role in cardiac hypertrophy in direct comparison with adiponectin's cardiac functions, mice deficient for either gene were subjected to TAC. Heart weight to body weight ratios were calculated. Deparaffinized sections from wild type, T-cadherin null and adiponectin null hearts were trichrome stained seven days after sham or TAC surgery.

No differences were observed in heart size between wild type and T-cadherin or adiponectin null mice in sham-operated controls (FIG. 4A). As expected, TAC induced compensatory hypertrophy in wild type hearts seven days after TAC. In T-cadherin and adiponectin null mice, the hypertrophic response was enhanced. Thus, a significant difference in heart weight to body weight ratio was noted in the T-cadherin and adiponectin null mice compared to wild type mice after TAC, with pressure overload significantly increasing heart size in T-cadherin null mice to a level comparable to adiponectin null hearts (FIG. 4B).

Echocardiographic data corroborated this finding. Echocardiography allows exact measurements of multiple parameters of cardiac architecture, such as the thickness of the heart wall at distinct locations within the heart both during systole and diastole. Diastolic wall thickness correlates with cardiac hypertrophy, while systolic wall dimensions yield insight into cardiac contractility. Diastolic left ventricular posterior wall thickness was quantified before and seven days after TAC surgery. M-mode echocardiograms were also performed in wild type, T-cadherin null, and adiponectin null mice seven days after TAC surgery. M-mode echocardiograms display the cardiac wall thickness as a function of time, and thus display systolic contractions.

No difference was found in diastolic left-ventricular posterior wall thickness (LVPWd) between the different groups after sham-operation. However, after TAC, T-cadherin null and adiponectin null mice showed significantly increased LVPWd compared to the wild type mice (FIGS. 4C, 4D, 15).

Since cardiac hypertrophy is the result of myofiber growth rather than cell proliferation, cardiomyocyte cross-sectional area in the free wall of the left ventricle was measured to confirm the echocardiography data on the single cell level. Wheat germ agglutination stained cell surfaces in hearts from wild type, T-cadherin null and adiponectin null mice were analyzed seven days after sham or TAC surgery. The free wall of the left ventricle and the papillary muscle, which are susceptible to pressure overload-induced stress, and thus commonly used for single cell analyses, were examined, and myocyte cross sectional areas were quantified. Cross-sectional area of cardiomyocytes was unchanged between the three genotypes after sham operation. Hypertrophy of T-cadherin null and adiponectin null cardiomyocytes was in accordance with the increased heart-to-body weight ratios and LVPWd measurements (FIGS. 4E, 4F). The wild type hearts showed significantly increased myofiber area after TAC, but the area increase was significantly larger in T-cadherin and adiponectin null hearts compared to the wild type hearts.

Together, these data implicate T-cadherin in anti-hypertrophic functions in the heart and indicate that compared to their wild type counterparts, T-cadherin and adiponectin null mice have a stronger hypertrophic response to pressure overload.

### Example 6

### AMPK Phosphorylation is Reduced in T-cadherin and Adiponectin Null Hearts after TAC

AMPK is a key regulator of cardiac metabolism, and its activation by stressors such as ischemia and increased AMP/ATP ratio inhibits energy-consuming processes such as protein synthesis. Adiponectin is known to mediate its cardioprotective and anti-hypertrophic function at least in part by phosphorylation of AMPK, which activates the kinase. Previous reports show reduced AMPK activity in adiponectin null hearts after TAC (11).

The phosphorylation of Thr-172 of the AMPKα subunit 1/2 was probed using a phospho-specific antibody. Wild type, T-cadherin null and adiponectin null myocardial lysates were immunoblotted seven days after TAC with phospho-specific (Thr-172) and total anti-AMPK, and anti-β-tubulin antibodies. AMPK phosphorylation was significantly reduced in T-cadherin null and adiponectin null hearts, indicating a role of T-cadherin in adiponectin-induced signaling and suggesting that the same signaling pathways are activated by both T-cadherin and adiponectin (FIGS. 5A, 5B).

### Example 7

### Decreased Survival in T-cadherin and Adiponectin Null Mice after TAC

Pressure overload-induced cardiac hypertrophy typically leads to dilated cardiomyopathy and reduced cardiac function. Cardiac function was determined by measuring fractional shortening analyzed using echocardiographic measurements in wild type, T-cadherin null and adiponectin null mice. The functional parameter of fractional shortening was significantly reduced in wild type mice seven days after TAC, but only a slight and non-significant reduction in fractional shortening was observed in T-cadherin and adiponectin null mice (FIG. 6A).

Previous reports have suggested that loss of adiponectin leads to increased mortality in mice after TAC (11). The mortality of T-cadherin null mice was compared with that of wild type and adiponectin null controls after TAC. The survival curve in FIG. 6B plots all mice which died during the first three to seven days after TAC. All animals that were found dead displayed extensive bleeding into the thoracic cavity, which is suggestive of aortic rupture. FIG. 6B shows that both T-cadherin and adiponectin null cohorts displayed increased and comparable mortality at seven days after TAC. Together with the preserved heart function in T-cadherin and adiponectin null mice, these data suggest that the decreased survival was not caused by heart failure. Rather, it suggests that the observed decrease in survival may involve changes in the aortic walls that render the vessels more susceptible to rupture during conditions of increased pressure. Under baseline conditions, no overt morphological differences were found in the thoracic aortas from wild type and T-cadherin and adiponectin null mice with regard to wall thickness and circumference. However, immunostaining for the macrophage-specific F4/80 antigen in sections from paraffin embedded wild type, T-cadherin null and adiponectin null aortas revealed increased macrophage invasion into the tunica media of T-cadherin and adiponectin null aortas compared to the wild type aortas (FIGS. 6C, 6D).

### Example 8

### Adiponectin Binds to the Intact Pro-Peptide Form of T-cadherin in the Mammary Gland and Heart

Western blots were performed in wild type, T-cadherin null and adiponectin null mice to determine which form(s) of T-cadherin are expressed in the mammary gland and heart in the presence and absence of endogenous adiponectin.

In wild type mice, T-cadherin was expressed in both the pro-peptide and mature forms (i.e., bands at about 120 kD and about 100 kD) in the mammary gland and heart (FIGS. 8, 9). However, in adiponectin null mice, T-cadherin was expressed only in the mature form (i.e., a band at about 100 kD) in the mammary gland and heart (FIGS. 8, 9). These data indicate that in the absence of adiponectin, the pro-peptide form of T-cadherin is cleaved to yield only the mature form of T-cadherin. These data further suggest that adiponectin may bind to the pro-peptide form of T-cadherin—crucial information for the methods, compositions and transgenic models described herein.

These combined findings suggest that pressure overload in T-cadherin and adiponectin null mice leads to two distinct phenomena: a) cardiomyocyte growth due at least in part to reduced protective AMPK activity, and b) rupture of mildly inflamed, and thus compromised, aortas due to increased wall stress. In addition, the similar phenotypes in T-cadherin and adiponectin null mice support a joint function of the two molecules.

### Example 9

### adAPN Restores T-cadherin Expression in Adiponectin Null Hearts

The findings provided herein suggest that T-cadherin interactions with adiponectin mediate cardioprotection. Adenovirus-expressed recombinant APN (adAPN) reverts the exacerbated cardiac hypertrophy of adiponectin null mice. As T-cadherin expression levels are low in adiponectin null mice (FIGS. 2A, 2C), the findings provided herein predict a mechanism by which adAPN upregulates T-cadherin expression in the myocardium to dock adAPN to cardiomyocytes.

To test this model, adAPN treatment was investigated for restoration of T-cadherin protein expression in adiponectin null hearts. Adiponectin null mice were injected with 2×10⁸ p.f.u. adAPN or GFP-expressing control adenovirus (adGFP) through the tail vein and were sacrificed after 10 days. Only animals with physiological concentrations of circulating adiponectin (i.e., within 2-fold of wild type levels) were included in the analyses (FIG. 16). adGFP-treated adiponectin null mice expressed low background cardiac T-cadherin protein levels as comparable to FIG. 2A (FIG. 13A). Treatment with adAPN yielded robust T-cadherin re-expression by cardiomyocytes in the adiponectin null mice, and ectopically expressed adAPN co-localized with T-cadherin (FIG. 13A). Cardiac T-cadherin mRNA levels remained unchanged in adiponectin null mice irrespective of the treatment, consistent with the notion that T-cadherin regulation occurs posttranslationally (FIG. 13B).

### Example 10

### Adiponectin Mediated Activation of the AMPK Signaling Pathway

To test the functional interaction between T-cadherin and adiponectin in limiting cardiac hypertrophy, mice deficient in both T-cadherin and adiponectin gene expression (“Tcad/APN dKO”) were generated. This was necessary, as T-cadherin null mice display high adiponectin serum levels (FIG. 2D) that would confound the interpretation of adAPN supplementation experiments. adAPN or adGFP virus was injected into adiponectin null and Tcad/APN dKO mice three days before TAC surgery, and animals were sacrificed seven days postsurgery.

Adiponectin null and Tcad/APN dKO mice treated with adGFP showed exaggerated cardiac hypertrophy after TAC, as observed in prior experiments with adiponectin null and T-cadherin null mice; there was no additive effect from the double mutation (FIGS. 13C-F). As expected, adAPN-treatment neutralized the exaggerated hypertrophy after TAC in adiponectin null mice (FIGS. 13C-F), and hearts showed only a modest hypertrophic response compared to the sham condition (FIG. 13D). In contrast, adAPN treatment of Tcad/APN dKO animals failed to reverse exaggerated cardiac hypertrophy, and hearts remained enlarged similar to those of control adGFP-treated adiponectin null and Tcad/APN dKO mice (FIGS. 13C-F). To corroborate these findings, the activation of AMPK was investigated seven days after TAC. In adAPN-treated adiponectin null mice, cardiac AMPK phosphorylation was enhanced compared to adGFP treated controls (FIGS. 13G, 13H). In contrast, there was no increase in phospho AMPK in the Tcad/APN dKO condition. To confirm this activating phosphorylation of AMPK, phosphorylation of Acetyl-CoA-carboxylase (ACC), which is a substrate of AMPK, was analyzed. ACC showed corresponding phosphorylation in adAPN-treated adiponectin null mice, while no increases were observed in the Tcad/APN dkO mice (FIGS. 13G, 131). These data suggest that T-cadherin limits cardiac hypertrophy through adiponectin mediated activation of the AMPK signaling pathway.

### Example 11

### Screening Candidate Compounds in Cardiomyocyte Cells

A panel of candidate compounds is selected to screen for a compound with the ability to confer a cardioprotective effect. Each candidate compound is introduced to a population of cardiomyocyte cells and analyzed to determine whether it is capable of stimulating the activity of a T-cadherin protein. Candidate compounds that generate increased signal transduction downstream of T-cadherin are identified as capable of stimulating the activating of a T-cadherin protein. Compounds identified during the screening can be used for further screening assays or methods of treatment as disclosed herein.

### Example 12

### Screening Candidate Compounds for Interaction with a Pro-peptide Form of T-cadherin

A panel of candidate compounds is selected to screen for a compound that interacts with a pro-peptide form of T-cadherin. Each candidate compound is introduced to the pro-peptide form of T-cadherin and analyzed to determine the presence of binding. Candidate compounds identified as binding to the pro-peptide form of T-cadherin can be used for further screening assays or methods of treatment as disclosed herein.

### Example 13

### Screening Candidate Compounds in a Mammal

A panel of candidate compounds is selected to screen for a compound with the ability to confer a cardioprotective effect. Each candidate compound is introduced to a mouse and analyzed to determine whether it is capable of stimulating the activity of a T-cadherin protein. Candidate compounds that decrease exacerbated heart size, heart weight to body weight ratio, LVPWd, myofiber area, macrophage infiltration, or the level of a pro-inflammatory marker are identified as capable of stimulating the activating of a T-cadherin protein. Compounds identified during the screening can be used for further screening assays or methods of treatment as disclosed herein.

### Example 14

### Conferring a Cardioprotective Effect in a Human

A human in need of a cardioprotective effect is identified and administered a compound known to interact with a pro-peptide form of T-cadherin. The human is monitored for a cardioprotective effect resulting from administration of the compound, such as the prevention, amelioration or reversal of cardiac hypertrophy, aortic rupture, or myocardial infarct. A cardioprotective effect is observed following administration of the compound.

### Example 15

### Modulating Cardiomyocyte Growth in a Human

A human at risk of a condition associated with cardiac pressure overload is identified and administered a compound known to interact with the pro-peptide portion of T-cadherin. Following administration of the compound, it is determined that the compound modulated cardiomyocyte growth in the human.

### Example 16

### Treating or Ameliorating a Cardiovascular Condition in a Human

A human in need of treatment for or amelioration of a cardiovascular condition is identified and administered a compound known to bind to or sequester a pro-peptide form of T-cadherin at the surface of cells. The human is monitored for a cardiovascular condition, such as the prevention, amelioration, or reversal of cardiac hypertrophy, aortic rupture, or myocardial infarct. Improvement in or amelioration of the cardiovascular condition is observed following administration of the compound.

### Summary of Methods

### Transverse Aortic Constriction (TAC)

8 to 12 week old male C57/B16 wild type, T-cadherin null or adiponectin null mice were shaved and anesthetized by intraperitoneal injection of ketamine (100 mg/kg) and xylazine (10 mg/kg). A first incision was made to expose the trachea and the mouse was intubated and ventilated using a volume cycled rodent ventilator. A second incision was made along the sternum and the chest cavity was entered through the second intercostal space at the left upper sternal border by blunt dissection. The thymus was carefully moved out of the field of view and the aortic arch was exposed to isolate the transverse aorta. Aortic constriction was achieved by tying a 7-0 nylon suture ligature against a 26-27 gauge needle and the aorta. The needle was subsequently removed yielding a remaining aortic diameter of about 0.4 mm at the site of the constriction. In adult mice, the aortic diameter is typically 1.0 to 1.2 mm in diameter. The described method of constriction leads to pressure gradients across the constriction of 30-60 mmHg. This is a sufficient stimulus to induce cardiac hypertrophy but does not lead to acute heart failure in normal mice. After TAC, the chest was closed with sutures and the pneumothorax was evacuated. Following extubation and the subcutaneous administration of the analgesic buprenophine (0.1-0.3 mg/kg) the mice were allowed to recover. Animals were closely monitored for signs of discomfort or shortness of breath. Sham operated mice underwent the same surgical procedure without constriction of the aorta.

### Echocardiography

Baseline echocardiography was done before TAC surgery and seven days after TAC before mice were sacrificed. Mice were anesthetized with isoflurane (1.25% in 100% oxygen). This level of anesthesia does not suppress spontaneous respiration and mechanical ventilation was not required. Echocardiography is an ultrasound method that allows for non-invasive examination of cardiac morphology and left ventricular function. An echocardiograph from VisualSonics was used. M-mode imaging was done at the level of the papillary muscle.

### Immunohistochemistry

Heart sections were deparaffinized, incubated with Target Retrieval Solution (Dako, Denmark) and pretreated with 1% Saponin (Fluka, Buchs, Switzerland) in PBS with 1 mM EGTA. Autofluorescence was quenched with saturated Sudan Black (MP Biomedicals, Santa Ma, Calif.) solution in 70% ethanol and sections were blocked with Antibody Diluent (Dako, Denmark). Sections were then incubated with adiponectin (PA1-054, Affinity Bioreagents, Golden, Colo.) or T-cadherin antibodies (18). Alexa 488 and Alexa 594 fluorescent conjugates (MOLECULAR PROBES®, Invitrogen, Carlsbad, Calif.) were used to detect the respective primary antibodies. Negative controls were slides processed in parallel without primary antibody. For T-cadherin and adiponectin antibodies, tissues from KO animals were used as additional controls.

### Histology

Deparaffinized heart sections were alternatively used for Trichrome staining. Frozen heart sections were fixed in acetone and processed for staining with fluorescently labeled wheat germ agglutinin (Sigma Aldrich, St. Louis, Mo.).

### Western Blotting

Snap frozen heart tissue was mechanically dissociated with mortar and pestle and suspended in lysis buffer (50 mmol/L Tris HCl pH 7.4, 150 mmol/L NaCl, 5 mmol/L EDTA, 1 mmol/L DTT, 1/100 protease inhibitor cocktail (SIGMA Aldrich, St. Louis, Mo.), 0.1 mmol/L phenylmethylsulfonyl fluoride and 1% NP40). Proteins (10 μg per lane) were separated by reducing SDS-PAGE and transferred to PVDF membranes. Membranes were then incubated with specific antibodies to adiponectin (PA1-054, Affinity Bioreagents, Golden, Colo.), T-cadherin (18), AdipoR1 (Abcam, Cambridge, Mass.), AdipoR2 (Phoenix Pharmaceuticals, Burlingame, Calif.), β-Tubulin (Chemicon, Temecula, Calif.), phospho-AMPKα (Thr172) (Cell Signaling, Danvers, Mass.), AMPKα (Cell Signaling, Danvers, Mass.), phospho-Acetyl CoA Carboxylase (Ser79) (Upstate Biotechnology Inc., Charlottesville, Va.), Acetyl CoA Carboxylase (Upstate, Charlottesville, Va.) and detected with HRP conjugated secondary antibodies (GE Healthcare, Piscataway, N.J.) using the ECL Detection Kit (Amersham, Amersham, UK).

### ELISA

In order to detect adiponectin in mouse serum, the APN-ELISA kit from R&D Systems (Minneapolis, Minn.) was used according to the manufacturer's recommendations.

### Mice

C57B1/6 Tcad-KO mice were previously described (18). Genotypes were determined by PCR. TcadFOR 5′-CTCTGAACAGGTAGTCGATAGCG-ACAGAC-3′ (SEQ ID NO:9) and TcadREV 5′-CGGAGACACTGCCTGTGTTCTCATTG-3′ (SEQ ID NO:10) amplified the wild type 120 by DNA fragment. TcadNEO 5′-GCATCGCCTTCTATCGCCTTCTTG-3′ (SEQ ID NO:11) and TcadFOR amplified the 350 by KO DNA fragment. C57B1/6 APN-KO mice were generated in Dr. Yuji Matsuzawa's laboratory (Osaka University, Osaka). APNfor 5′-GGAACTTGTGCAGGTTGGAT-3′ (SEQ ID NO:12) and APNrev 5′-CAGTGCAAGCTCCAAGATGA-3′ (SEQ ID NO:13) amplified the WT 266 by DNA fragment. APNKOfor 5′-ATACTTTCTCGGCAGGAGCA-3′ (SEQ ID NO:14) and APNrev amplified the 900 by KO DNA fragment. A lack of APN protein in APN-KO animals was confirmed by ELISA (R&D, Minneapolis, Minn.). Animals were fed with a normal diet.

### Quantitative Real-Time PCR

Total RNA was extracted from cardiac tissue using Trizol (Invitrogen, Carlsbad, Calif.). Equal amounts were reverse transcribed using oligo (dT)₁₈ and random hexamer primers (Transcriptor First Strand cDNA Synthesis Kit, Roche, Pleasanton, Calif.). Real-time PCR analysis was performed with SYBR green using the Stratagene Mx3000p instrument. T-cadherin (5′-catcgaagctcaagatatgg-3′ (SEQ ID NO:1); 5′-gatttccattgatgatggtg-3′) (SEQ ID NO:2), AdipoR1 (5′-acacagagactggcaacatc-3′(SEQ ID NO:3); 5′-gagcaatccctgaatagtcc-3′ (SEQ ID NO:4)), and AdipoR2 (5′-tggacacatctcctaggttg-3′ (SEQ ID NO:5); 5′-tagagaagagtcgggagacc-3′ (SEQ ID NO:6)) cDNAs were detected and normalized to GAPDH (5′-ccagtatgactccactcacg-3′ (SEQ ID NO:7); 5′-gactccacgacatactcagc-3′ (SEQ ID NO:8)).

### Adenovirus-Mediated Gene Transfer

2×108 plaque-forming units (p.f.u.) of adAPN11 or adGFP (generated in Dr. M. Mercola's laboratory, Sanford-Burnham Medical Research Institute, La Jolla, USA) were injected into the tail vein of mice three days prior to TAC. GFP fluorescence was verified in the liver. APN was detected in the circulation using Western blotting and ELISA.

### Statistical Analysis

Statistical analyses were done in Prism. All statistical analyses were done by one-way analysis of variance (ANOVA) with Bonferroni post-tests, thus significance levels were *P<0.05; **P<0.01; ***P<0.001 and values are mean±s.e.m

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

### REFERENCES

1. Lorell B H, Carabello B A. Left ventricular hypertrophy: pathogenesis, detection, and prognosis. Circulation 2000; 102: 470-9.
2. Croquelois A, Domenighetti A A, Nemir M. et al. Control of the adaptive response of the heart to stress via the Notch I receptor pathway. J Exp Med 2008:205: 3173-85.
3. Molkentin J D. Calcineurin-NFAT signaling regulates the cardiac hypertrophic response in coordination with the MAPKs. Cardiovasc Res 2004; 63: 467-75.
4. Shiojima I, Walsh K. Regulation of cardiac growth and coronary angiogenesis by the Akt/PKB signaling pathway. Genes Dev 2006; 20: 3347-65.
5. Kemi O J, Ceci M, Wisloff U, et al. Activation or inactivation of cardiac Akt/mTOR signaling diverges physiological from pathological hypertrophy. J Cell Physio12008; 214: 316-21.
6. Sano M, Minamino T, Toko H. et al. p53-induced inhibition of Hif-I causes cardiac dysfunction during pressure overload. Nature 2007; 446: 444-8.
7. Dyck J R, Lopaschuk G D. AMPK alterations in cardiac physiology and pathology: enemy or ally? J PhysioI2006; 574: 95-112.
8. Shibata R, Ouchi N, Ito M. et al. Adiponectin-mediated modulation of hypertrophic signals in the heart. Nat Med 2004; 10: 1384-9.
9. Okamoto Y, Kihara S, Funahashi T, Matsuzawa Y, Libby P. Adiponectin: a key adipocytokine in metabolic syndrome. Clin Sci (Lond) 2006; 110: 267-78.
10. Hopkins T A, Ouchi N, Shibata R, Walsh K. Adiponectin actions in the cardiovascular system. Cardiovasc Res 2007; 74: 11-8.
11. Kudrjashova E, Bashtrikov P, Bochkov V, et al. Expression of adhesion molecule T-cadherin is increased during neointima formation in experimental restenosis. Histochem Cell Biol 2002; 118: 281-90.
12. Matsumoto H, Tamura S, Kamada Y, et al. Adiponectin deficiency exacerbates lipopolysaccharide/D-galactosamine-induced liver injury in mice. World J Gastroenterol 2006; 12; 3352-8.
13. Goldstein R. I., Scalia R G, Ma X L. Protective vascular and myocardial effects of adiponectin. Nat Clin Pract Cardiovasc Med 2009; 6: 27-35.
14. Doyle D D, Goings G E, Upshaw-Earley J, Page E, Ranscht B, Palfrey He. T-cadherin is a major glycophosphoinositol-anchored protein associated with noncaveolar detergent-insoluble domains of the cardiac sarcolemma. J Biol Chem 1998; 273: 6937-43.
15. Takahashi T, Saegusa S, Sumino H, et al. Adiponectin, T-cadherin and tumour necrosis factor-alpha in damaged cardiomyocytes from autopsy specimens. J Int Med Res 2005; 33: 236-44.
16. Chien et al. 1989.
17. Vestal D J, Ranscht B. Glycosyl phosphatidylinositol-anchored T-cadherin mediates calcium-dependent, homophilic cell adhesion. J Cell Biol 1992; 119(2): 451-61.
18. Hebbard L W, Garlatti M, Young L.1, Cardiff R D, Oshima R G, Ranscht B. T-cadherin supports angiogenesis and adiponectin association with the vasculature in a mouse mammary tumor model. Cancer Res 2008; 68: 1407-16.
19. Lam J B, Chow K H, Xu A, Lam K S, Liu J, Wong N S, Moon R T, Shepherd P R, Cooper G J, Wang Y. Adiponectin haploinsufficiency promotes mammary tumor development in MMTV-PyVT mice by modulation of phosphatase and tensin homolog activities. PLoS One 2009; 4(3) e4968.
20. Yamauchi T, Kamon J, Ito Y. et al. Cloning of adiponectin receptors that mediate antidiabetic metabolic effects. Nature 2003; 423: 762-9.
21. Gollan L, Sabanay H, Poliak S, Berglund E O, Ranscht B, Peles E. Retention of a cell adhesion complex at the paranodal junction requires the cytoplasmic region of Caspr. J Cell Biol 2002; 157: 1247-56.
22. Lim Y S, McLaughlin T, Sung Te, Santiago A, Lee K F, O'Leary D D. p75(NTR) mediates ephrin-A reverse signaling required for axon repulsion and mapping. Neuron 2008; 59: 746-58.
23. Shibata, R., et al. Adiponectin protects against myocardial ischemia-reperfusion injury through AMPK- and COX-2-dependent mechanisms. Nat Med 11, 1096-1103 (2005).
24. Lingwood D, Simons K.2010 Lipid rafts as a membrane-organizing principle. Science. 2010 Jan. 1; 327(5961):46-50.
25. Biochem Soc Symp. 2005;(72):151-64. Consequences of lipid raft association on G-protein coupled receptor function. Becher A, Mclhlinney R A.
26. Targeting of ion channels to membrane microdomains: localization of KV channels to lipid rafts. Martens JR, O'Connell K, Tamkun M. Trends Pharmacol Sci. 2004 January;25(1):16-21.
27. Involvement of lipid rafts and caveolae in cardiac ion channel function./// Maguy A, Hebert T E, Nattel S. Cardiovasc Res. 2006 Mar. 1; 69(4):798-807. Epub 2006 Jan. 6.
28. Curr Opin Nephrol Hypertens. 2007 March; 16(2):90-104. Vascular signaling through cholesterol-rich domains: implications in hypertension. Callera G E, Montezano A C, Yogi A, Tostes R C, Touyz R M.

What is claimed is:

1. A method of conferring a cardioprotective effect in a mammal, comprising administering a compound to said mammal, wherein said compound is known to interact with a pro-peptide form of T-cadherin.

2. The method of claim 1, wherein said interaction comprises the binding of said compound to said pro-peptide form of T-cadherin.

3. The method of claim 1, wherein said interaction comprises the sequestration of said pro-peptide form of T-cadherin to the surface of a cell in said mammal.

4. The method of claim 1, wherein said cardioprotective effect comprises the prevention, amelioration or reversal of a condition selected from the group consisting of: cardiac hypertrophy, aortic rupture, and myocardial infarct.

5. A method of modulating cardiomyocyte growth, comprising: administering to a subject a compound known to interact with the pro-peptide portion of T-cadherin.

6. A method of treating or ameliorating a cardiovascular condition, comprising administering a compound known to bind to a pro-peptide form of T-cadherin.

7. The method of claim 6, wherein said cardiovascular condition is selected from the group consisting of: cardiac hypertrophy, aortic rupture, and myocardial infarct.

8. A method of screening a candidate compound for the ability to confer a cardioprotective effect, said method comprising:
providing a candidate compound to a cell; and
determining whether said candidate compound is capable of stimulating the activity of a T-cadherin protein.

9. The method of claim 8, wherein determining whether said candidate compound is capable of stimulating the activity of a T-cadherin protein comprises measuring the level of AMP-activated protein kinase (AMPK) phosphorylation in said cell.

10. The method of claim 8, wherein said candidate compound interacts with a pro-peptide form of said T-cadherin protein.

11. The method of claim 8, wherein said candidate compound interacts with a mature form of said T-cadherin protein.

12. The method of claim 8, wherein said candidate compound is a compound from a peptide library.

13. The method of claim 8, wherein said cell is a cardiomyocyte cell.

14. The method of claim 8, wherein said cardioprotective effect comprises the prevention, amelioration or reversal of cardiac hypertrophy in a mammal.

15. The method of claim 14, wherein said cardiac hypertrophy has been detected or predicted using a measurement in heart size.

16. The method of claim 14, wherein said cardiac hypertrophy has been detected or predicted using a measurement in heart weight to body weight ratio.

17. The method of claim 14, wherein said cardiac hypertrophy has been detected or predicted using a measurement in diastolic left-ventricular posterior wall thickness (LVPWd).

18. The method of claim 14, wherein said cardiac hypertrophy has been detected or predicted using a measurement in myofiber area.

19. The method of claim 8, wherein said cardioprotective effect comprises the prevention, amelioration or reversal of aortic rupture in a mammal.

20. The method of claim 19, wherein said aortic rupture has been detected or predicted using a measurement in macrophage infiltration into the aortic wall.

21. The method of claim 19, wherein said aortic rupture has been detected or predicted using a measurement in a pro-inflammatory marker.

22. The method of claim 8, wherein said cardioprotective effect comprises the prevention, amelioration or reversal of myocardial infarct in a mammal.

23. The method of claim 8, wherein said compound is an adiponectin polypeptide or fragment thereof.

24. The method of claim 8, further comprising determining whether said test compound is capable of, or likely to be capable of, conferring a cardioprotective effect in said cell.

25. A method of identifying a molecule that transduces an adiponectin signal to the interior of a cell comprising:
providing a candidate molecule to a cell; and
determining whether said candidate molecule is capable of stimulating the activity of T-cadherin, wherein if said candidate molecule is capable of stimulating the activity of T-cadherin it is identified as transducing an adiponectin signal.

26. The method of claim 25, wherein said determining a stimulated activity of said T-cadherin protein comprises detecting a posttranslational modification of said T-cadherin protein.

27. A method of treating cardiac hypertrophy in a mammal, said method comprising identifying a mammal suffering from cardiac hypertrophy and providing to said mammal a therapeutically effective amount of an adiponectin polypeptide or fragment thereof.

28. A method of treating aortic rupture in a mammal, said method comprising identifying a mammal suffering from aortic rupture and providing to said mammal a therapeutically effective amount of an adiponectin polypeptide or fragment thereof.

29. A method for the treatment and/or prophylaxis of a condition characterized by cardiac hypertrophy in a mammal, said method comprising modulating the interaction of adiponectin and T-cadherin in a mammal, wherein upregulating the activity of said adiponectin to a functionally effective level upregulates said interaction of adiponectin and T-cadherin in said mammal.

30. The method of claim 29, wherein said modulation is achieved by introducing to said mammal an adiponectin polypeptide or fragment thereof.

31. The method of claim 30, wherein said adiponectin polypeptide or fragment thereof interacts with a pro-peptide form of said T-cadherin protein.

32. The method of claim 30, wherein said adiponectin polypeptide or fragment thereof interacts with a mature form of said T-cadherin protein.

33. The method of claim 29, wherein said condition is selected from the group consisting of: myocardial infarct, hypertension, aortic stenosis, ischemic heart disease, valvular insufficiency, infectious agents or mutations in sarcomeric genes.

34. A method for the treatment and/or prophylaxis of a condition characterized by aortic rupture in a mammal, said method comprising modulating the interaction of adiponectin and T-cadherin in a mammal, wherein upregulating the activity of adiponectin to a functionally effective level upregulates said interaction of adiponectin and T-cadherin.

35. The method of claim 34, wherein said modulation is achieved by introducing to said mammal an adiponectin polypeptide or fragment thereof.

36. The method of claim 34, wherein said compound interacts with a pro-peptide form of said T-cadherin protein.

37. The method of claim 34, wherein said compound interacts with a mature form of said T-cadherin protein.

38. The method of claim 34, wherein said condition is selected from the group consisting of: myocardial infarct, hypertension, aortic stenosis, ischemic heart disease, valvular insufficiency, infectious agents or mutations in sarcomeric genes.

39. A genetically modified mouse comprising a disrupted T-cadherin gene, wherein said genetically modified mouse is homozygous for said disrupted T-cadherin gene, and wherein said genetically modified mouse exhibits an increase in cardiac hypertrophy compared to a mouse that does not have said disrupted T-cadherin gene.

40. The mouse of claim 39, wherein said disrupted T-cadherin gene comprises a tissue-specific null mutation.

41. The mouse of claim 39, wherein said increase in cardiac hypertrophy is detected using a measurement in heart size.

42. The mouse of claim 39, wherein said increase in cardiac hypertrophy is detected using a measurement in heart weight to body weight ratio.

43. The mouse of claim 39, wherein said increase in cardiac hypertrophy is detected using a measurement in diastolic LVPWd.

44. The mouse of claim 39, wherein said increase in cardiac hypertrophy is detected using a measurement in myofiber area.

45. The mouse of claim 39, wherein said increase in cardiac hypertrophy is detected using a measurement in macrophage infiltration into the aortic wall.

46. The mouse of claim 39, wherein said increase in cardiac hypertrophy is detected using a measurement in a pro-inflammatory marker.

47. A genetically modified mouse comprising a disrupted adiponectin gene and a disrupted T-cadherin gene, wherein said genetically modified mouse is homozygous for said disrupted adiponectin gene and said disrupted T-cadherin gene.

48. The mouse of claim 47, wherein said genetically modified mouse exhibits an increase in cardiac hypertrophy compared to a mouse that does not have said disrupted adiponectin gene or said disrupted T-cadherin gene.

49. A method of using a genetically modified mouse to identify a compound that confers a cardioprotective effect comprising:
providing the genetically modified mouse of claim 39;
contacting said genetically modified mouse with a compound;
determining whether said compound is capable of stimulating the activity of a T-cadherin protein; and
for a candidate compound capable of stimulating said activity of T-cadherin, determining whether said candidate compound is capable of, or likely to be capable of, conferring a cardioprotective effect in said mouse.

50. The method of claim 49, further comprising subjecting said mouse to transaortic constriction (TAC) to determine whether said candidate compound is capable of, or likely to be capable of, conferring said cardioprotective effect in said mouse.

51. A method of using a genetically modified mouse to identify a compound that confers a cardioprotective effect comprising:
providing the genetically modified mouse of claim 47;
contacting said genetically modified mouse with a compound;
determining whether said compound is capable of stimulating the activity of a T-cadherin protein; and
for a candidate compound capable of stimulating said activity of T-cadherin, determining whether said candidate compound is capable of, or likely to be capable of, conferring a cardioprotective effect in said mouse.

52. The method of claim 51, further comprising subjecting said mouse to TAC to determine whether said candidate compound is capable of, or likely to be capable of, conferring said cardioprotective effect in said mouse.

53. The method of claim 51, wherein said determining a stimulated activity of said T-cadherin protein comprises detecting a posttranslational modification of said T-cadherin protein.

54. A composition capable of stimulating the activity of a T-cadherin protein, wherein said composition comprises an adiponectin polypeptide or fragment thereof.

55. The composition of claim 54, wherein said composition comprises a region capable of binding with a pro-peptide form of said T-cadherin protein.

56. The composition of claim 54, wherein said composition comprises a region capable of binding with a mature form of said T-cadherin protein.

57. A composition for the prevention, amelioration or reversal of a cardiovascular condition, wherein said composition is configured to upregulate a posttranslational modification of T-cadherin.

58. The composition of claim 57, wherein said cardiovascular condition is selected from the group consisting of: cardiac hypertrophy, aortic rupture, and myocardial infarct.
